(19) United States
(12) Patent Application Publication
(10) Pub. No.: US 2026/0253669 A1
(43) Pub. Date: Aug. 27, 2026

(54) Variant Identification in the Presence of Alignment Artifacts

(71) Applicants: The Broad Institute, Inc., Cambridge, MA (US); Massachusetts Institute of Technology

(72) Inventors: Victoria Popic, Somerville, MA (US); Megan Le, Cambridge, MA (US); Bonnie Berger, Cambridge, MA (US)

(73) Assignees: The Broad Institute, Inc., Cambridge, MA (US); Massachusetts Institute of Technology, (US)

(21) Appl. No.: 19/548,140

(22) Filed: Feb. 24, 2026

Related U.S. Application Data
(60) Provisional application No. 63/762,260, filed on Feb. 24, 2025.

(51) Int. Cl. G16B 20/20; G16B 30/10; G16B 45/00

(52) U.S. Cl. CPC G16B 20/20; G16B 30/10; G16B 45/00

(57) ABSTRACT

Variant identification in the presence of alignment artifacts is described. Discordant reads of sequencing data may be identified with respect to a reference sequence. A variant-indicative signal may be generated based at least in part on a subread analysis of the discordant reads, the variant-indicative signal including one or both of a discordant junction signal or an alignment generated using a latent breakpoint graph. A variant call may be generated based at least in part on the variant-indicative signal, the variant call identifying a structural variant in the sequencing data as compared to the reference sequence.

### RELATED APPLICATION

This application claims priority to U.S. Provisional Patent Application Ser. No. 63/762,260, filed Feb. 24, 2025, entitled “Variant Identification in the Presence of Alignment Artifacts,” the entire disclosure of which is hereby incorporated by reference herein in its entirety.

### STATEMENT REGARDING GOVERNMENT SUPPORT

This invention was made with government support under Grant Nos. HG012467 and GM141861 awarded by the National Institutes of Health. The government has certain rights in the invention.

### BACKGROUND

Genomic structural variants (SVs) are large-scale alterations in the genome, typically defined as changes of fifty base pairs or larger. These variants include deletions, duplications, inversions, translocations, and other complex rearrangements. SVs are a major source of genetic diversity among individuals and play important roles in evolution, disease susceptibility, and phenotypic variation.

The detection and characterization of SVs has traditionally relied on short-read sequencing technologies. However, short reads present challenges for accurately identifying SVs, particularly in repetitive regions of the genome. Short reads often fail to span entire SV events or complex genomic structures, leading to incomplete or inaccurate variant calls. This limitation has hindered comprehensive SV discovery and genotyping across diverse genome types.

Long-read sequencing technologies have emerged as a promising approach for improved SV detection. The increased read lengths allow for spanning of repetitive regions and entire SV events. However, long-read technologies introduce new computational challenges for SV discovery algorithms. Systematic alignment artifacts are prevalent in long-read data and can confound the signals used to detect SVs.

Systematic reference bias may also impair variant calling, as the alignment process may favor matches to the reference sequence over true variants. While pangenomes have been proposed as a solution to reference bias, they may have limitations in capturing the full spectrum of human genetic diversity.

### SUMMARY

Variant identification in the presence of alignment artifacts is described. Discordant reads of sequencing data may be identified with respect to a reference sequence. A variant-indicative signal may be generated based at least in part on a subread analysis of the discordant reads, the variant-indicative signal including one or both of a discordant junction signal or an alignment generated using a latent breakpoint graph. A variant call may be generated based at least in part on the variant-indicative signal, the variant call identifying a structural variant in the sequencing data as compared to the reference sequence.

This Summary introduces a selection of concepts in a simplified form that are further described below in the Detailed Description. As such, this Summary is not intended to identify essential features of the claimed subject matter, nor is it intended to be used as an aid in determining the scope of the claimed subject matter.

### BRIEF DESCRIPTION OF THE DRAWINGS

The detailed description is described with reference to the accompanying figures.

FIG. 1 is an illustration of an environment in an example implementation that is operable to employ variant identification in the presence of alignment artifacts as described herein.

FIG. 2 depicts an example implementation of the subread analysis module introduced in FIG. 1 in greater detail.

FIG. 3 depicts an example implementation of the latent breakpoint graph analysis module of FIG. 1 in greater detail.

FIG. 4 depicts an overview of alignment artifacts that may occur due to a deletion structural variant in a sample sequence.

FIG. 5 depicts an overview illustrating an example subread alignment corresponding to a deletion structural variant.

FIG. 6 depicts an overview illustrating an example subread alignment corresponding to a duplication structural variant.

FIG. 7 depicts an overview illustrating an example subread alignment corresponding to an inversion structural variant.

FIG. 8 depicts an overview illustrating an example latent breakpoint graph analysis.

FIG. 9 depicts an example procedure in which structural variant identification is performed.

FIG. 10 depicts an example procedure for generating a variant-indicative signal using a subread analysis.

FIG. 11 depicts an example procedure for generating a variant-indicative signal using a latent breakpoint graph analysis.

FIG. 12 illustrates an example system including various components of an example device that can be implemented as any type of computing device as described and/or utilized with reference to FIGS. 1-11 to implement the techniques described herein.

### DETAILED DESCRIPTION

### Overview

Structural variant (SV) identification refers to the systematic analysis and detection of large-scale alterations in an individual's genetic material, particularly in a deoxyribonucleic acid (DNA) sequence. This process involves identifying and characterizing genomic rearrangements that are typically 50 base pairs or larger, including deletions, duplications, inversions, translocations, and other complex rearrangements. SV identification aims to unveil sources of genetic diversity among individuals, which contributes to understanding evolution, disease susceptibility and/or progression, and phenotypic variation.

For example, bioinformatics techniques can be applied to process and analyze genomic data to determine qualitative and quantitative information about structural variants. Genomic data for SV identification is typically generated via DNA sequencing techniques. For instance, short-read sequencing techniques may be used to determine sequences of nucleotide bases in a DNA sample obtained from a subject, producing fragments typically ranging from 50 to 500 base pairs. However, these short reads may present challenges for accurately identifying SVs, particularly in repetitive regions of the genome, because short reads often fail to span entire SV events or complex genomic structures.

Long-read sequencing has emerged as a promising approach for improved SV detection. Long-read sequencing techniques produce sequence fragments typically greater than 10,000 base pairs, such as in a range from several kilobases to over a megabase. These extended read lengths may allow for spanning of repetitive regions and entire SV events. However, because even long reads are a fraction of the human genome, these sequence fragments are mapped (e.g., aligned) with a reference genomic sequence in a process referred to as alignment.

The genomic sequence of the sample may be compared to the reference genomic sequence to determine structural variants in a process referred to as variant calling. SV calling includes identifying and characterizing large-scale genetic variations in the genomic sequence of the sample in comparison to the reference genomic sequence. These variants can include copy number variants (CNVs, where a segment of DNA ranging from kilobases to megabases in size is duplicated or deleted), inversions (e.g., a segment of DNA is reversed in orientation), large insertions or deletions involving segments of more than 50 nucleotides, and translocations (e.g., where a segment of DNA moves from one location to another, often involving the exchange of genetic material between non-homologous chromosomes). Small variant calling includes identifying and characterizing smaller genetic variations in the genomic sequence of the sample in comparison to the reference sequencing, including single nucleotide polymorphisms (SNPs) and short insertions and deletions (indels) of 50 nucleotides or less.

Traditional alignment techniques may face several challenges in detecting SVs. Short reads frequently fail to span entire SV events, leading to incomplete or inaccurate variant calls. While long-read sequencing technologies may produce reads that better capture SV events, long-read data may introduce new computational challenges. Moreover, alignment artifacts may occur in both short-and long-read alignments due to an optimization objective used in read alignments. Systematic alignment artifacts may confound the signals used to detect SVs. For example, traditional alignment techniques employ objective functions that bias toward continuous, ungapped alignments. This contiguity bias can cause misalignment of reads at portions including structural variants, potentially extending the alignment with mismatches and forcing local alignment rather than breaking the contiguity.

These alignment artifacts may impact downstream variant discovery of both structural variants and small (e.g., short) variants. For example, common systematic alignment artifacts may include artifacts at deletion sites, duplication sites, and inversion sites. At deletion sites, for example, reads may not be split to cross the deletion and may instead map into the deletion site. This leads to loss of the deletion SV signal and false positive small SNP calls. In some scenarios, the reads are clipped, leading to further loss of signal. As another example, at inversion sites, the reads may not be split and may instead map as an insertion and deletion of similar lengths (e.g., a deletion length is approximately equal to an insertion length, which is further approximately equal to the inversion length) and/or as many mismatches to the reference, leading to false positive SNP/indel calls. As yet another example, at duplication sites, the reads may include an insertion of size equal to the duplication instead of being split.

As may be appreciated from the above discussion, structural variants may be overlooked or mischaracterized as regions having many small variants (e.g., false positive SNP and indel calls). Accordingly, the limitations of current alignment methods can lead to incomplete or inaccurate detection of structural variants and/or small variants, which may hinder comprehensive variant discovery and genotyping across diverse genome types. However, an accurate identification of variants may enhance understanding of genome evolution and/or disease etiology and/or may guide personalized medicine.

To overcome these problems, variant identification in the presence of alignment artifacts is disclosed herein. In one or more implementations, sequencing data of a sample may be aligned to a reference sequence to generate an initial alignment. Reads of the sequencing data that map discordantly to the reference sequence (e.g., “discordant reads”) may be identified based on the initial alignment at areas having a plurality of mismatches to the reference sequence, an insertion with respect to the reference sequence, a deletion with respect to the reference sequence, an inversion, split alignments, and/or clipped alignments. A variant-indicative signal may be generated based on the discordant reads and may include a discordant junction signal and/or a graph alignment generated using a latent breakpoint graph.

In one or more implementations, the sequencing data may include long-read sequencing data, where each read may include greater than 1000 base pairs and may range up to over a megabase in length. A sequencing run for a biological sample may produce a coverage depth of tens to hundreds of reads spanning each position in a genome comprising billions of base pairs. The analysis of such sequencing data to detect discordant reads, partition the discordant reads into subreads, map the subreads to detect novel adjacencies, and generate variant calls based on the novel adjacencies involves a volume and complexity of data that is not practically performed by manual human analysis.

In at least one implementation, a subread analysis method is used, where the discordant reads are partitioned into subreads that are mapped to corresponding portions of the reference sequence. The subreads may range from about 50 to 1000 base pairs in length, allowing for more precise local alignments. Non-contiguous alignments (e.g., novel adjacencies), such as subreads that are adjacent (e.g., consecutive) in the original sequence but map to opposite strands or out of order, may be detected and indicated as discordant junctions used for the discordant junction signal. The discordant junction signal may include information about the type, location, and characteristics of the detected novel adjacencies and may provide candidate SV junctions to a structural variant caller, for example.

In at least one variation, alternatively or in addition, a latent breakpoint graph analysis method may be used, where a latent breakpoint graph is constructed based on the discordant junctions identified via the subread analysis. The latent breakpoint graph may be a graph of nodes representing genomic segments from the reference sequence and edges connecting the nodes, where a first set of edges represents adjacencies between the genomic segments in the reference sequence and a second set of edges represents novel adjacencies identified based on the discordant junctions. The latent breakpoint graph may also include user-defined edges to incorporate prior knowledge or focus the analysis on specific genomic regions of interest. The latent breakpoint graph represents potential genomic rearrangements, providing alternate paths between genomic segments compared to the reference sequence. The sequencing data may then be aligned to this graph to generate the updated alignment. The alignment process may use a scoring function that considers factors such as the number of matching bases, gaps, insertions, and deletions to determine the best scoring alignment path through the latent breakpoint graph for each read.

In one or more implementations, the techniques described herein circumvent the alignment biases of existing techniques in a flexible, computationally efficient manner. Whether using the subread analysis method directly or in conjunction with the latent breakpoint graph analysis method, the detection of novel adjacencies between genomic segments provides a robust signal for identifying various types of SVs, including inversions, translocations, and other complex rearrangements that may be missed by traditional alignment methods. As such, the techniques described herein may improve the accuracy and sensitivity of detecting both structural variants and small variants, thereby providing a more comprehensive picture of genomic variation.

Moreover, the alignment generated using the latent breakpoint graph analysis may be corrected for the alignment artifacts produced by traditional alignment techniques. Accordingly, the techniques described herein improve alignment system function by overcoming contiguity bias to provide more accurate alignments.

In some aspects, the techniques described herein relate to a method for structural variant identification, including: identifying discordant reads of sequencing data with respect to a reference sequence; generating a variant-indicative signal based at least in part on a subread analysis of the discordant reads, the variant-indicative signal including one or both of a discordant junction signal or an alignment generated using a latent breakpoint graph; and outputting a variant call based at least in part on the variant-indicative signal, the variant call identifying a structural variant in the sequencing data as compared to the reference sequence.

In some aspects, the techniques described herein relate to a method, wherein generating the variant-indicative signal based at least in part on the subread analysis of the discordant reads includes: partitioning the discordant reads into subreads; mapping the subreads to a corresponding portion of the reference sequence; detecting novel adjacencies between at least a portion of subread; and outputting the novel adjacencies as the discordant junction signal.

In some aspects, the techniques described herein relate to a method, wherein the novel adjacencies include a junction between a first subread and a second subread that map to locations on the reference sequence with a different relative distance compared to their respective positions in a corresponding read.

In some aspects, the techniques described herein relate to a method, wherein the novel adjacencies include a junction between a first subread and a second subread that map to the reference sequence in a different order as compared to a corresponding read.

In some aspects, the techniques described herein relate to a method, wherein the novel adjacencies include a junction between a first subread and a second subread that map to opposite strands of the reference sequence, while originating from a same strand in a corresponding read.

In some aspects, the techniques described herein relate to a method, wherein generating the variant-indicative signal includes: generating the latent breakpoint graph based at least in part on the discordant junction signal, the latent breakpoint graph including nodes representing genomic segments from the reference sequence and edges connecting the nodes, wherein: a first set of edges represents adjacencies between the genomic segments in the reference sequence, and a second set of edges represents novel adjacencies defined based on the discordant junction signal; and generating a graph alignment by aligning the sequencing data to the latent breakpoint graph.

In some aspects, the techniques described herein relate to a method, further including: receiving an initial alignment of the sequencing data mapped to the reference sequence; for each read of the sequencing data, selecting between the graph alignment and the initial alignment based on respective alignment scores; and generating the alignment by using a selected one of the graph alignment or the initial alignment for each read of the sequencing data.

In some aspects, the techniques described herein relate to a method, wherein generating the latent breakpoint graph is further based on user input, and wherein the user input defines a third set of edges between the nodes.

In some aspects, the techniques described herein relate to a method, wherein identifying the discordant reads of the sequencing data is based on an initial alignment of the sequencing data to the reference sequence, and wherein the initial alignment is a full alignment of the sequencing data to the reference sequence or a partial alignment of the sequencing data to the reference sequence.

In some aspects, the techniques described herein relate to a method, wherein identifying the discordant reads of the sequencing data includes performing a k-mer analysis by: dividing reads of the sequencing data into tokens; mapping the tokens to the reference sequence; and indicating a read of the sequencing data as discordant in response to the tokens of the read mapping to multiple strands of the reference sequence.

In some aspects, the techniques described herein relate to a system for structural variant identification, including: a processing system; and a computer-readable storage medium having instructions stored thereon that, when executed by the processing system, cause the processing system to perform operations including: identifying discordant reads of sequencing data obtained for a sample, the discordant reads including reads of the sequencing data that include at least one of a threshold number of mismatches to a reference sequence, an insertion with respect to the reference sequence, a deletion with respect to the reference sequence; generating a variant-indicative signal based at least in part on a subread analysis of the discordant reads, the subread analysis defining novel adjacencies in the discordant reads; and generating a variant call of the sample based at least in part on the variant-indicative signal, the variant call identifying a structural variant in the sequencing data as compared to the reference sequence.

In some aspects, the techniques described herein relate to a system, wherein generating the variant-indicative signal based at least in part on the subread analysis of the discordant reads includes: partitioning the discordant reads into subreads; mapping the subreads to a corresponding portion of the reference sequence; and detecting the novel adjacencies between at least a portion of the subreads based on at least one of: a change in a relative position of the subreads as compared to the reads; a change in an order of the subreads as compared to the reads; or an alignment of the subreads to different strands of the reference sequence.

In some aspects, the techniques described herein relate to a system, wherein generating the variant-indicative signal includes: generating a latent breakpoint graph based at least in part on the novel adjacencies by: defining nodes representing genomic segments from the reference sequence, defining a first set of edges between the nodes representing adjacencies between the genomic segments in the reference sequence, and defining a second set of edges between the nodes representing the novel adjacencies; and generating a graph alignment by aligning the sequencing data to the latent breakpoint graph.

In some aspects, the techniques described herein relate to a system, wherein the operations further include: generating an initial alignment by mapping the sequencing data to the reference sequence; and generating an updated alignment based on the graph alignment and the initial alignment by: for each read of the sequencing data, selecting one of the graph alignment or the initial alignment based on respective alignment scores; and including the selected one of the graph alignment or the initial alignment in the updated alignment.

In some aspects, the techniques described herein relate to a system, wherein the latent breakpoint graph further includes: a third set of edges between the genomic segments in the reference sequence defined by user input.

In some aspects, the techniques described herein relate to a method for structural variant identification, including: obtaining sequencing data of a sample; generating a variant-indicative signal based on a subread analysis of sequencing reads of the sequencing data, the subread analysis defining novel adjacencies in the sequencing reads; and generating a variant call based at least in part on the variant-indicative signal and a reference sequence, the variant call identifying a structural variant in the sequencing data as compared to the reference sequence.

In some aspects, the techniques described herein relate to a method, wherein generating the variant-indicative signal based on the subread analysis of the sequencing reads includes: partitioning the sequencing reads into subreads; mapping the subreads to a corresponding portion of the reference sequence; detecting the novel adjacencies between at least a portion of the subreads based on at least one of: a change in a relative position of the portion of the subreads compared to a corresponding original position in the sequencing reads; a change in an order of the portion of the subreads compared to a corresponding original order in the sequencing reads; or an alignment of the portion of the subreads to different strands of the reference sequence; and outputting the novel adjacencies as a discordant junction signal that includes at least part of the variant-indicative signal.

In some aspects, the techniques described herein relate to a method, wherein generating the variant-indicative signal includes: generating a latent breakpoint graph based at least in part on the novel adjacencies by: defining nodes representing genomic segments from the reference sequence; defining edges between the nodes, the edges representing adjacencies between the genomic segments in the reference sequence; and defining additional edges between the nodes based on the novel adjacencies; and generating a graph alignment by aligning the sequencing data to the latent breakpoint graph.

In some aspects, the techniques described herein relate to a method, further including: generating an initial alignment by aligning the sequencing data to the reference sequence; for each read of the sequencing data, selecting one of the graph alignment or the initial alignment based on respective alignment scores; and generating an updated alignment having the selected one of the graph alignment or the initial alignment for each read of the sequencing data.

In some aspects, the techniques described herein relate to a method, wherein generating the variant call includes: comparing the variant-indicative signal to the reference sequence to identify genomic differences between the sequencing data and the reference sequence; for each identified genomic difference: determining a type of variant based on a size and a type of the identified genomic difference, wherein the type of variant includes at least one of a deletion structural variant, an insertion structural variant, an inversion structural variant, a translocation structural variant, a duplication structural variant, a single-nucleotide polymorphism, or a short indel; and determining a genomic position of the identified genomic difference; and generating a report that includes, for each identified genomic difference, the type of variant, the genomic position, and the size.

In the following discussion, an example environment is first described that may employ the techniques described herein. Example implementation details and procedures are then described which may be performed in the example environment as well as other environments. Consequently, performance of the example procedures is not limited to the example environment and the example environment is not limited to performance of the example procedures.

### Example Environment

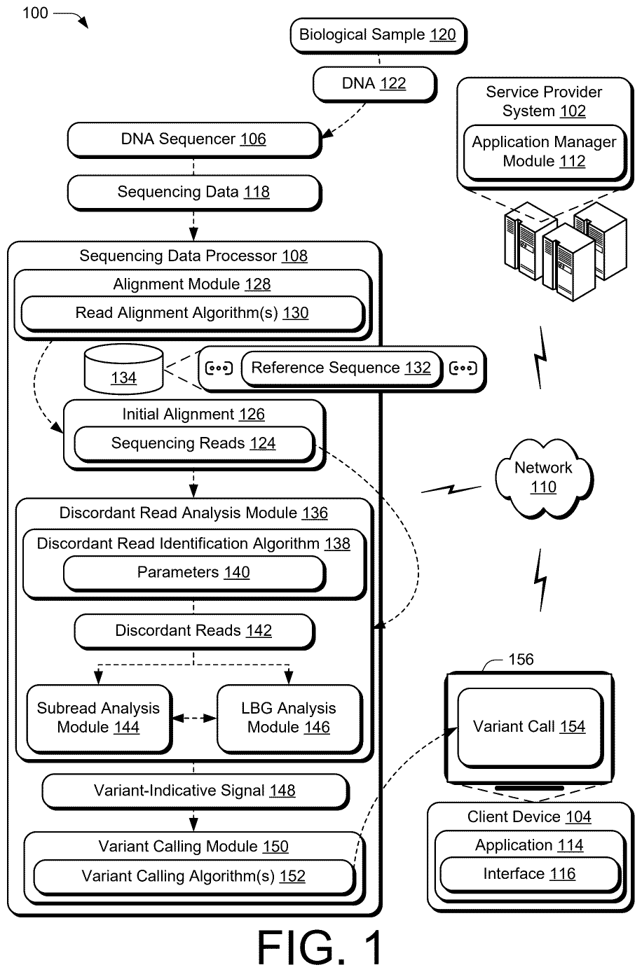

FIG. 1 is an illustration of an environment 100 in an example implementation that is operable to employ variant identification in the presence of alignment artifacts as described herein. The illustrated environment 100 includes a service provider system 102, a client device 104, a DNA sequencer 106, and a sequencing data processor 108 that are communicatively coupled, one to another, via a network 110. The network 110 may enable wired and/or wireless electronic communication, for example. Although the sequencing data processor 108 is illustrated as separate from the service provider system 102, the client device 104, and the DNA sequencer 106, this functionality may be incorporated as part of the service provider system 102, the client device 104, and/or the DNA sequencer 106, further divided among other entities, and so forth.

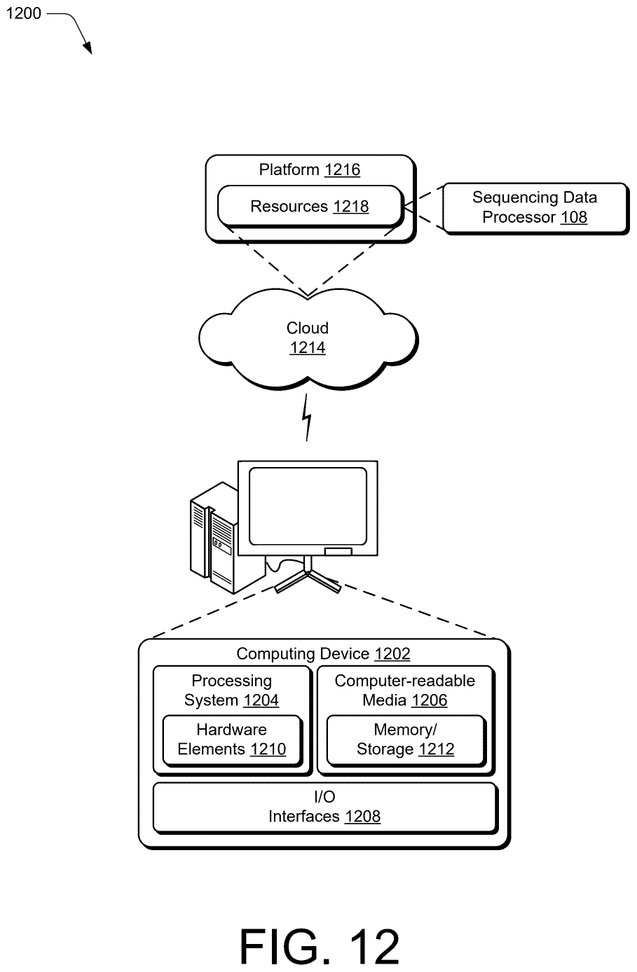

Computing devices that are usable to implement the service provider system 102, the client device 104, and the sequencing data processor 108 may be configured in a variety of ways. A computing device, for instance, may be configured as a desktop computer, a laptop computer, a mobile device (e.g., assuming a handheld configuration such as a tablet or mobile phone), and so forth. Thus, the computing device may range from full-resource devices with substantial memory and processor resources to a low-resource device with limited memory and/or processing resources. Additionally, a computing device may be representative of a plurality of different devices, such as multiple servers utilized to perform operations “over the cloud,” as will be further described in relation to FIG. 12.

The service provider system 102 is illustrated as including an application manager module 112 that is representative of functionality to provide access to the sequencing data processor 108 to a user of the client device 104 via the network 110. The application manager module 112, for instance, may expose content or functionality of the sequencing data processor 108 that is accessible via the network 110 by an application 114 of the client device 104. The application 114 may be configured as a network-enabled application, a browser, a native application, and so on, that exchanges data with the service provider system 102 via the network 110.

In the context of the described techniques, the application 114 includes functionality to analyze sequencing data to identify structural variants. The application 114 includes an interface 116 that is implemented at least partially in hardware of the client device 104 for facilitating communication between the client device 104 and the sequencing data processor 108. The interface 116 includes functionality to receive inputs to the sequencing data processor 108 from the client device 104 and output information, data, and so forth from the sequencing data processor 108 to the client device 104.

The DNA sequencer 106 is configured to produce sequencing data 118 that is analyzed by the sequencing data processor 108 to identify structural variants. The DNA sequencer 106 may use one of a plurality of sequencing techniques to produce the sequencing data 118, e.g., “sequencing reads” or “reads.” In at least one implementation, the DNA sequencer 106 uses a long-read sequencing technique that produces sequence fragments that typically range from approximately 1000 bases to 1,000,000 bases and more typically from 5000 bases to 900,000 bases in length. These sequence fragments are referred to as “long reads.” In at least one variation, however, short-read sequencing is used, which produces sequence fragments typically ranging from approximately 10 bases to approximately 1000 bases and more typically from approximately 50 bases to approximately 500 bases. Sequence fragments produced via short-read sequencing techniques are also referred to as “short reads.”

Sequencing, for instance, includes determining an order of nucleotides (e.g., adenine, thymine or uracil, cytosine, and guanine) in a sample of nucleic acids, such as nucleic acids derived from a biological sample 120. The order of nucleotides is referred to herein as a “sequence.” The nucleotides are also referred to as “bases.” Although the sequencing event will be described herein with respect to deoxyribonucleic acid (DNA) sequencing, it is to be appreciated that the techniques described herein may be adapted for sequencing other types of nucleic acids, such as the sequencing of complementary DNA (cDNA) derived from ribonucleic acid (RNA) transcripts.

In the illustrated example environment 100, the DNA sequencer 106 produces the sequencing data 118 from DNA 122 obtained from the biological sample 120. By way of example, the biological sample 120 may include cells (e.g., tissue or fluid) obtained from a subject (e.g., an individual, such as a patient, or another type of organism, such as a bacteria) and/or a culture, and the DNA 122 may be extracted from the biological sample 120.

In at least one implementation, the sequencing data 118 comprise a text-based file format, such as FASTQ files that store both nucleotide sequence information and quality scores for the bases in a sequencing read. In variations, the sequencing data 118 comprise another type of file format. The sequencing data 118 include sequencing reads 124. The sequencing data processor 108 is configured to receive the sequencing data 118 and determine an initial alignment 126 therefrom, e.g., as represented in a binary alignment/map (BAM) file, for instance. The BAM file may allow for subsequent variant analysis, as will be further elaborated below. In at least one implementation, the initial alignment 126 is a partial alignment where portions of the sequencing reads 124 are aligned to the reference sequence 132, such as will be further described below. In at least one variation, the initial alignment 126 is omitted.

In one or more implementations, the sequencing data processor 108 further includes an alignment module 128. The alignment module 128 is representative of functionality for determining the initial alignment 126 of the sequencing reads 124. Read alignment, also referred to simply as “alignment,” involves mapping nucleic acid segments to locations in the genome. The alignment module 128 is configured to receive the sequencing data 118 and align (e.g., map) the sequencing reads 124 to a reference sequence 132.

It is contemplated that the reference sequence 132 can be selected from a variety of nucleic acid sequences against which a sequence of a sample can be compared for determining variants of the sequence of the sample. The reference sequence 132 is a reference genome or a portion thereof. In one or more implementations, the sequencing data processor 108 includes or otherwise accesses a storage device 134 storing the reference sequence 132. The storage device 134 may store one or more other reference sequences in addition to the reference sequence 132, as indicated by ellipses in FIG. 1. By way of example, different reference sequences may correspond to different sample types or may come from a pangenome, which includes several high-quality, curated assemblies of individual genomes that may be represented jointly as a graph. A pangenome graph, for instance, is a computational data structure that represents the genetic variation present across multiple genomes of the same or related species. The pangenome graph may include nodes representing genomic sequences and edges representing connections between those sequences. By way of example, the nodes may represent conserved genomic regions or variant alleles that are known within the population, while edges may represent adjacencies between the genomic regions, including between the variant alleles. Paths through the graph may represent individual genomic sequences or haplotypes. This type of graph structure may also be used for latent breakpoint graphs (LBGs) used in variant identification, as will be elaborated herein.

As such, the reference sequence 132 is selected based on its similarity to the sample evaluated via the sequencing event, at least in some implementations. Moreover, the reference sequence 132 may include a combination of more than one individual reference sequence.

In at least one implementation, the alignment module 128 includes at least one read alignment algorithm 130 configured to map the sequencing reads 124 to the reference sequence 132 to generate the initial alignment 126. By way of example, the at least one read alignment algorithm 130 may include an objective function that aims to maximize the match between a given read of the sequencing reads 124 and the reference sequence 132 by penalizing mismatches and gaps (e.g., insertions or deletions) and preserving contiguity. In at least one implementation, the objective function causes the at least one read alignment algorithm 130 to bias toward continuous, ungapped alignments (referred to herein as “contiguity”), which may cause the at least one read alignment algorithm 130 to misalign the sequencing reads 124 at portions including structural variants. By way of example, the at least one read alignment algorithm 130 may be configured to extend the alignment with mismatches, forcing local alignment rather than breaking the contiguity of the alignment. As a result, structural variants may be overlooked or mischaracterized as a region having a great many single nucleotide polymorphisms or indels, for example.

Accordingly, the sequencing data processor 108 further includes a discordant read analysis module 136. The discordant read analysis module 136 is representative of the functionality for analyzing discordantly aligned sequencing reads 124 as a part of identifying variants. In at least one implementation, the discordant read analysis module 136 extracts an additional data signal that may be used to identify structural variants that may otherwise be suppressed by the at least one read alignment algorithm 130. The additional data signal, for instance, may be otherwise obscured by alignment artifacts. Alternatively, or in addition, the discordant read analysis module 136 may implement a multi-step process to overcome the limitations of traditional alignment algorithms with respect to the complex genomic rearrangements that can occur in structural variants.

In at least one implementation, the discordant read analysis module 136 first identifies the sequencing reads 124 from the initial alignment 126 that exhibit signs of improper mapping, such as large insertions or stretches of mismatches, using a discordant read identification algorithm 138. The discordant read identification algorithm 138 may include parameters 140 to identify discordant reads 142 (e.g., the sequencing reads 124 that exhibit signs of improper, or “discordant,” mapping with the reference sequence 132). At least a portion of the discordant reads 142 may encode variants, including structural variants, and may exhibit poor mapping due to alignment artifacts (e.g., “artifacts”) caused by the at least one read alignment algorithm 130, such as described above. The parameters 140, for instance, may include configurable (e.g., user-configurable) parameters used by the discordant read identification algorithm 138, such as a minimum insertion or deletion size, a minimum (e.g., threshold) number of mismatches within a configurable range of base pairs, a change in sequencing depth, and/or a threshold for a ratio of mismatches to matches within the configurable range. The parameters 140 may be adjusted to fine-tune the sensitivity and/or specificity of the discordant read detection based on characteristics of the sequencing data 118 (e.g., a sequencing technique used) and/or user preferences. As such, the discordant reads 142 may be sequencing reads 124 having significant differences from the reference sequence 132 (e.g., many mismatches, many small indels, at least one large indel, split alignments, and/or clipped alignments, collectively referred to herein as “alignment characteristics”) as defined by the parameters 140. By allowing for customization of the parameters 140, the discordant read analysis module 136 may provide flexibility in adapting to different sequencing technologies, genome complexities, and research objectives.

Alternatively, or in addition, the discordant read identification algorithm 138 may identify the discordant reads 142 based on a partial alignment and/or k-mer analysis of the sequencing reads 124. In at least one implementation, this analysis bypasses the initial alignment step (e.g., by the at least one read alignment algorithm 130) and processes sample sequences directly from the sequencing data 118 (e.g., FASTQ file). The partial alignment, for instance, may align a beginning portion (e.g., prefix) and an end portion (e.g., suffix) of a given sequencing read 124 to the reference sequence 132. If a distance between the beginning portion and the end portion of the given sequencing read 124 when aligned to the reference sequence 132 is significantly different (e.g., at least a threshold difference, as defined by the parameters 140) than within the read itself, then the given sequencing read 124 is indicated as discordant by the discordant read identification algorithm 138. By way of example, the distance may be significantly different due to a copy number variant (e.g., a deletion or a duplication) or a novel insertion. For the k-mer analysis, the discordant read identification algorithm 138 may divide the given sequencing read 124 into smaller tokens (e.g., k-mers of “k” consecutive nucleotides, where k is defined by the parameters 140) and use k-mer identity-based matching to efficiently map the k-mers to the reference sequence 132. In response to the tokens of a given read mapping to multiple strands of the reference sequence 132, which may be a result of an inversion structural variant, the given read may be indicated as discordant.

In one or more implementations, reads that are indicated as discordant based on the alignment characteristics are confirmed to be discordant reads 142 by fully mapping the corresponding read(s) to the reference sequence 132 using the at least one read alignment algorithm 130, such as described above. In at least one variation, however, reads that are indicated as discordant are assumed to be the discordant reads 142 without confirming/verifying via the full alignment process. The partial alignment and k-mer-based approaches described above may identify discordant reads 142 for further analysis by the discordant read analysis module 136 with reduced time and computational resources compared to generating the full alignment.

In at least one variation, the discordant read analysis module 136 may input substantially all of the sequencing reads 124 to the downstream analysis modules described herein. By way of example, the discordant read analysis module 136 may process all or substantially all of the sequencing reads 124 as the discordant reads 142 without first checking for discordance with respect to the reference sequence 132. Doing so may decrease computational efficiency but may increase variant detection sensitivity, for example.

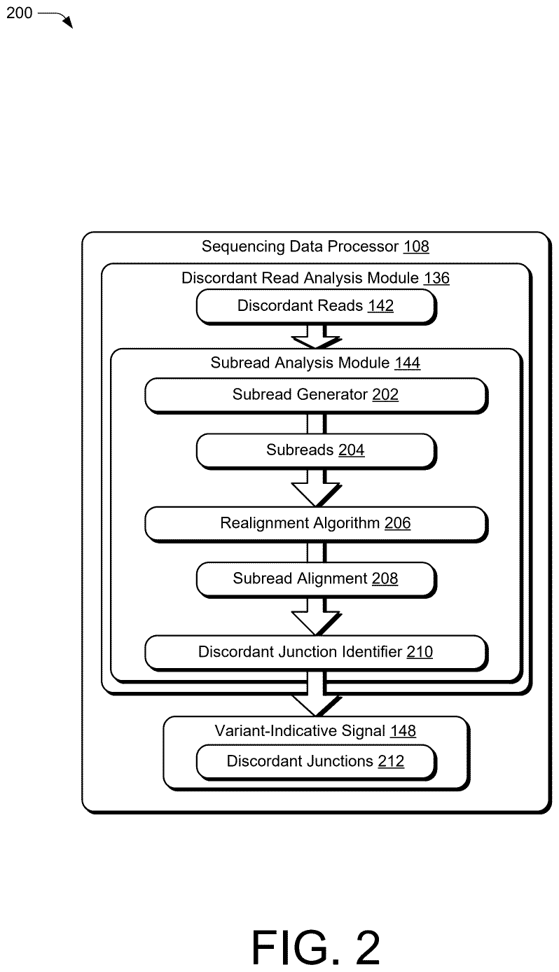

The discordant read analysis module 136 may further evaluate the discordant reads 142 using one or both of a subread analysis module 144 and a latent breakpoint graph (LBG) analysis module 146 to generate a variant-indicative signal 148. By way of example, the subread analysis module 144 may represent the functionality of the discordant read analysis module 136 to generate and align (e.g., realign) subreads of the discordant reads 142, as will be further described below with respect to FIG. 2. In at least one variation, the subread analysis module 144 may generate and align subreads for substantially all of the sequencing reads 124.

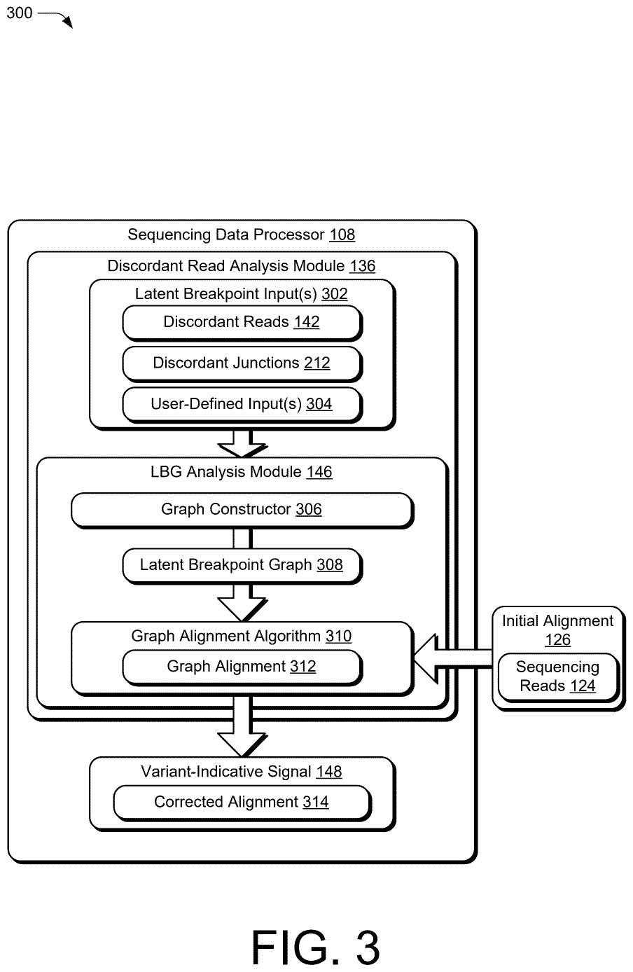

The LBG analysis module 146 may represent the functionality of the discordant read analysis module 136 to construct a latent breakpoint graph. The latent breakpoint graph may be a personalized graph representation of the genomic structure of the biological sample 120 based on evidence derived from the sequencing reads 124, including the discordant reads 142 and the subread analysis module 144. The LBG analysis module 146, for instance, may use information derived from the discordant reads 142 to add edges representing possible/candidate (e.g., “latent”) novel genomic segment adjacencies and generate a new and/or updated alignment, as will be further described with respect to FIG. 3.

In at least one implementation, the LBG analysis module 146 uses information from the subread analysis module 144 as input for identifying the latent breakpoints and adds edges representing candidate adjacencies given by the subread analysis module. Whether used alone or in combination, the subread analysis module 144 and the LBG analysis module 146 may identify novel adjacencies that may indicate potential structural rearrangement breakpoints, which are locations where structural variations may occur.

It is to be appreciated that although the discordant read analysis module 136 is shown including both the subread analysis module 144 and the LBG analysis module 146, in at least one variation, the discordant read analysis module 136 includes only the subread analysis module 144 or only the LBG analysis module 146.

The discordant read analysis module 136 is shown outputting the variant-indicative signal 148. In at least one implementation, the variant-indicative signal 148 comprises discordant junctions and/or non-contiguous alignments identified through the subread analysis process of the subread analysis module 144. Alternatively, or in addition, the variant-indicative signal 148 comprises novel adjacencies and/or structural rearrangements captured by the latent breakpoint graph generated by the LBG analysis module 146, as will be further described herein. Alternatively, or in addition, the variant-indicative signal 148 does not include the full alignment information, and only includes the type of novel adjacency and the breakpoints in the genome. By way of example, the variant-indicative signal 148 may indicate novel adjacencies, which may be used directly for variant calling. The variant-indicative signal 148, for instance, may indicate a novel adjacency signal for each read of the sequencing reads 124. In reads where the discordant read analysis module 136 does not identify a novel adjacency, the novel adjacency signal may indicate that no novel adjacency is present. In at least one other variation, alternatively or in addition, the variant-indicative signal 148 directly reports a graph alignment generated by the LBG analysis module 146 for each read of the sequencing reads 124, such as when the initial alignment 126 is not generated.

The variant-indicative signal 148 may be passed to a variant calling module 150. Although not explicitly shown in FIG. 1, in at least one implementation, the variant calling module 150 further receives the initial alignment 126. The variant calling module 150 may be representative of the functionality for determining genomic differences (e.g., variants) between the sequencing reads 124 and the reference sequence 132. By way of example, at least a portion of the variants may be detected by identifying positions where the initial alignment 126 differs from the reference sequence 132. In one or more implementations, the variant calling module 150 may include one or more variant calling algorithms 152, which may be used to generate a variant call 154. In one or more implementations, the variant calling module, in addition to or instead of alignment information, may call variants directly from the type and breakpoint position of the novel adjacencies provided by the subread analysis module. The variant call 154 may include an indication of short variants, such as single nucleotide polymorphisms (SNPs, where one nucleotide is changed to another at a specific position in the genome), as well as structural variants that are determined to be present in the sequencing reads 124 as compared to the reference sequence 132, based at least in part on the variant-indicative signal 148. In at least one implementation, the variant call 154 may include information about the type, location, and characteristics of the identified variants. This call may serve as a summary of the genomic differences detected by the one or more variant calling algorithms 152. The types of structural variants reported by the variant call 154 may include deletions, duplications, inversions, translocations, insertions, and complex structural variants. For example, deletions include the loss of a segment of DNA, duplications include extra copies of a DNA segment, inversions occur when a segment of DNA is reversed in orientation, translocations include the movement of DNA from one location to another, and insertions add new DNA sequences into the genome (e.g., as compared to the reference sequence 132).

In one or more implementations, the variant call 154 may be used to inform clinical decision-making. For example, the variant call 154 may indicate structural variants associated with a disease condition, a predisposition to a disease condition, or a predicted response to a therapeutic intervention. A clinician may use the variant call 154 to determine, adjust, or select a treatment plan for a subject from whom the biological sample 120 was obtained. By way of example, the variant call 154 may identify a structural variant associated with a cancer, and the treatment plan may be adjusted based on the identified structural variant. As another example, the variant call 154 may identify a structural variant associated with a pharmacogenomic response, and a drug selection or dosage may be determined based on the identified structural variant. In one or more implementations, the variant call 154 may be used in research applications to identify structural variants associated with phenotypic traits or disease mechanisms. By way of example, the variant call 154 may be used to identify structural variants that contribute to a heritable condition, to understand disease etiology, and/or to characterize genomic rearrangements in a population study.

The client device 104 is shown displaying, via a display device 156, the variant call 154. It is to be appreciated that the variant call 154, the sequencing data 118, the initial alignment 126, and/or the discordant reads 142 may be also stored in a memory of the sequencing data processor 108 and/or the client device 104 for subsequent access. By way of example, the variant call 154, the sequencing data 118, the initial alignment 126, and/or the discordant reads 142 may be stored in a single data file or in multiple data files. It is to be appreciated that at least a portion of the information in the variant call 154, the sequencing data 118, the initial alignment 126, and/or the discordant reads 142 may be stored in a standardized file format, e.g., tab-separated values (TSV), to facilitate and automate downstream processing.

As such, the artifact detection and realignment process performed by the discordant read analysis module 136 may significantly enhance the detection of structural variants in the sequencing data 118. By leveraging the inherent advantages of long-read sequencing for mapping repetitive regions of the genome while mitigating the alignment artifacts that may suppress structural variant identification via the discordant read analysis module 136, a more comprehensive and accurate picture of genomic structural variation may be provided. Moreover, false positive short variants may be reduced and/or avoided.

Accordingly, by reducing false positive small variant calls and improving the detection of structural variants that may otherwise be missed or mischaracterized due to alignment artifacts, the techniques described herein may provide more reliable information for clinical and research applications. By way of example, a structural variant that is missed or mischaracterized as a cluster of single nucleotide polymorphisms may result in a missed diagnosis, a less effective treatment selection, or a failure to identify a clinically actionable genomic alteration. By detecting structural variants that may otherwise be obscured by alignment artifacts, the techniques described herein may enable identification of clinically relevant genomic alterations that would otherwise go undetected, which may impact diagnosis, treatment selection, and patient outcomes. FIG. 2 depicts an example implementation 200 of the subread analysis module 144 introduced in FIG. 1 in greater detail.

In at least one implementation, the subread analysis module 144 may partition the discordant reads 142 (e.g., as identified via the discordant read identification algorithm 138 of FIG. 1) into shorter fragments via a subread generator 202. The subread generator 202 may divide a given discordant read 142 into subreads 204 of a configurable length. The subreads 204, for instance, may be shorter segments of the discordant read 142, particularly when the discordant reads 142 include long-read sequencing data. The subreads 204, for instance, may range from 50 to 800 base pairs in length, depending on a configuration of the subread analysis module 144. The subreads 204 may allow for more precise local alignments with the reference sequence 132 compared to the full-length discordant reads 142. The subread analysis module 144 may then realign the subreads 204 to the reference sequence 132, or a portion thereof, using a realignment algorithm 206, generating a subread alignment 208.

The subread analysis module 144 further includes a discordant junction identifier 210, which receives the subread alignment 208. The discordant junction identifier 210 is representative of the functionality of the subread analysis module 144 to identify subreads 204 of the subread alignment 208 that map to the reference sequence 132 with novel adjacencies (e.g., non-contiguous alignments). These novel adjacencies form discordant junctions 212 compared to the adjacencies of the subreads 204 in the corresponding discordant read 142. Thus, as used herein, the term “discordant junction” refers to a junction formed between pairs of consecutive subreads 204 derived from a single discordant read 142 that map to the reference sequence 132 with novel adjacencies (e.g., non-contiguous alignments). Discordant junctions 212 may form between pairs of subreads 204 that map to opposite strands of the reference sequence, pairs of subreads 204 that map in an unexpected order relative to their original positions in the discordant read 142, and pairs of subreads 204 that map to non-contiguous regions of the reference sequence 132. The discordant junctions 212 may serve as indicators of structural variants, particularly for complex rearrangements such as inversions, translocations, and duplications that may be missed or mischaracterized by the at least one read alignment algorithm 130 of FIG. 1.

In some implementations, the discordant junction identifier 210 may use a sliding window approach to scan the subread alignment 208, identifying regions where the subread alignment 208 deviates from the expected linear progression of the corresponding discordant read 142 and/or portion of the reference sequence 132. Alternatively, or in addition, the discordant junction identifier 210 may employ statistical methods to consider factors such as mapping quality, coverage depth, and the frequency of similar junction patterns across multiple reads to identify the discordant junctions 212.

In the implementation 200, the discordant junctions 212 identified by the discordant junction identifier 210 may be output as at least a part of the variant-indicative signal 148. This data may be passed to the variant calling module 150, as described in FIG. 1, for further analysis and generation of the variant call 154. As such, in at least one implementation, the discordant junctions 212 provide an additional signal used by the variant calling module 150 along with, or instead of, the initial alignment 126 to output the variant call 154, as further elaborated herein.

In one or more implementations, the subread analysis module 144 transforms the discordant reads 142 into the variant-indicative signal 148 by partitioning the discordant reads 142 into the subreads 204 and detecting the novel adjacencies between the subreads 204. The discordant reads 142, which may appear as regions of mismatches or insertions when aligned as full-length reads to the reference sequence 132, are thereby transformed into a signal indicating structural rearrangement breakpoints. The variant-indicative signal 148 represents a different data structure than the discordant reads 142, encoding the locations and types of the discordant junctions 212 rather than raw sequence data.

FIG. 3 depicts an example implementation 300 of the LBG analysis module 146 of FIG. 1 in greater detail.

The LBG analysis module 146 receives latent breakpoint input(s) 302, which may include the discordant reads 142 and/or the discordant junctions 212 from the subread analysis module. For example, the discordant junctions 212 may indicate latent edges. In at least one implementation, the input(s) 302 further include user-defined input(s) 304, allowing for customization of the analysis process based on specific research objectives or known genomic characteristics of the sample. By way of example, the user-defined input(s) 304 may include experimentally determined and/or theoretical breakpoints between genomic regions, such as potential boundaries of jumping genes or other mobile genetic elements. The user-defined input(s) 304 may allow the breakpoint analysis process to be customized, to focus on specific areas of interest, and/or to incorporate prior knowledge about the genomic structure being analyzed. In some instances, the user-defined input(s) 304 may help refine the detection of complex structural variants.

In the implementation 300, the LBG analysis module 146 includes a graph constructor 306, which generates a latent breakpoint graph 308 based on the provided inputs. The latent breakpoint graph 308 provides a personalized representation of the genomic structure of the biological sample 120, capturing potential structural variations and rearrangements. By way of example, the latent breakpoint graph 308 comprises nodes representing genomic segments and edges representing potential adjacencies between these segments, including both known adjacencies from the reference sequence 132 and novel adjacencies inferred from the discordant reads 142 and/or the discordant junctions 212. In at least one implementation, the edges further include custom adjacencies from the user-defined input(s) 304.

A “breakpoint,” for example, refers to a specific location in the genome where a structural rearrangement has occurred, resulting in a discontinuity or change in the linear sequence of nucleotides compared to the reference sequence 132. Breakpoints may be associated with various types of structural variants, including deletions, insertions, inversions, and translocations. Accordingly, a “latent breakpoint” may refer to a potential genomic breakpoint that is inferred from sequencing data but has not been directly observed or confirmed. These latent breakpoints are represented as edges in the latent breakpoint graph 308, indicating possible structural variations in the DNA 122 of the biological sample 120 and providing possible paths through the graph.

In at least one implementation, the graph constructor 306 generates the latent breakpoint graph 308 by defining nodes representing genomic segments from the reference sequence and edges between the nodes as the paths through the graph. By way of example, the graph constructor 306 may define a first set of edges representing adjacencies between the genomic segments in the reference sequence 132 and a second set of edges representing the latent breakpoints corresponding to novel adjacencies (e.g., as determined directly from the discordant reads 142 and/or based on the discordant junctions 212). In at least one implementation, the graph constructor 306 further defines edges between the nodes based on the user-defined input(s) 304 (e.g., a third set of edges).

In at least one implementation, the latent breakpoint graph 308 represents a transformed data structure derived from the reference sequence 132 and the discordant junctions 212. Unlike the reference sequence 132, which represents a linear or pangenomic representation of genomic segments, the latent breakpoint graph 308 includes edges representing novel adjacencies that are specific to the biological sample 120. The latent breakpoint graph 308 may thus provide a sample-specific representation of potential genomic rearrangements that is not present in the reference sequence 132 or in standard pangenome references. This transformed graph structure enables alignment paths that would not be possible using the reference sequence 132 alone, thereby allowing the graph alignment algorithm 310 to identify alignments that better explain the observed sequencing data 118.

Once the latent breakpoint graph 308 is constructed, a graph alignment algorithm 310 is employed to align (e.g., realign) the sequencing reads to this personalized graph structure. The graph alignment algorithm 310 is representative of the functionality of the LBG analysis module 146 to align the sequencing reads 124 to the latent breakpoint graph 308, generating a graph alignment 312. In at least one implementation, the graph alignment algorithm 310 compares each sequencing read 124 against the latent breakpoint graph 308, considering both the standard reference paths and the novel adjacencies introduced by the latent breakpoints (and/or the user-defined input(s) 304) to identify the best-mapping path using a scoring function. This approach allows for a more comprehensive exploration of possible alignments, particularly in regions where structural variants may be present. By doing so, the graph alignment 312 may include alignments that better explain the observed sequencing reads 124 compared to the initial alignment 126, especially in regions where the initial alignment 126 to the reference sequence 132 may have been suboptimal (e.g., due to the discordant reads 142).

In at least one implementation, the graph alignment algorithm 310 compares the alignment scores of the sequencing reads 124 in the graph alignment 312 with their scores from the initial alignment 126. The alignment with the better score for a given sequencing read 124 is selected for inclusion in a corrected alignment 314. the corrected alignment 314 includes the graph alignment 312.

In the implementation 300, the corrected alignment 314 is output as at least part of the variant-indicative signal 148, which can be further analyzed by the variant calling module 150 (as described in FIG. 1) to generate the variant call 154.

In one or more implementations, the corrected alignment 314 represents a transformed data structure that differs structurally and functionally from the initial alignment 126. For example, the corrected alignment 314 may include split-read representations for reads that were previously represented as contiguous alignments with mismatches in the initial alignment 126. As such, the corrected alignment 314 may have a reduced number of mismatches compared to the initial alignment 126, a reduced number of false positive single nucleotide polymorphism indications, and an increased number of split-read alignments that accurately represent structural variant breakpoints. The transformation from the initial alignment 126 to the corrected alignment 314 may result in a data structure that more accurately represents the genomic structure of the biological sample 120, thereby enabling more accurate downstream variant calling for both structural variants and small variants. In some aspects, the corrected alignment 314 may be stored in a modified alignment file format, such as a BAM file, that includes additional metadata indicating the graph-based realignment and the novel adjacencies identified through the subread analysis.

In this way, both structural variants and small variants may be identified with increased accuracy and sensitivity. Via the implementation 200 of FIG. 2, for example, the subreads 204 may enable more precise local alignments and identification of the discordant junctions 212, which are indicative of novel adjacencies missed by traditional alignment methods and standard references (e.g., the single linear reference or the pangenome reference). As another example, via the implementation 300 of FIG. 3, the latent breakpoint graph 308 may provide a personalized genomic structure reference for the graph alignment 312, which may capture a wider range of potential structural variations than a standard pangenome reference or a pangenome reference augmented with standard augmentation techniques.

By using these approaches alone or in combination, the techniques described herein may overcome the limitations of standard alignment algorithms (e.g., the at least one read alignment algorithm 130), particularly with respect to objective functions that favor contiguity, to achieve more accurate variant identification of both structural variants and smaller variants such as single nucleotide polymorphisms. By improving alignment system function through correction of alignment artifacts, the techniques described herein may reduce missed diagnoses and improve treatment selection based on the resulting variant calls.

Having discussed example details of the techniques for variant identification in the presence of alignment artifacts, consider now an example to illustrate usage of the techniques.

### Example Application

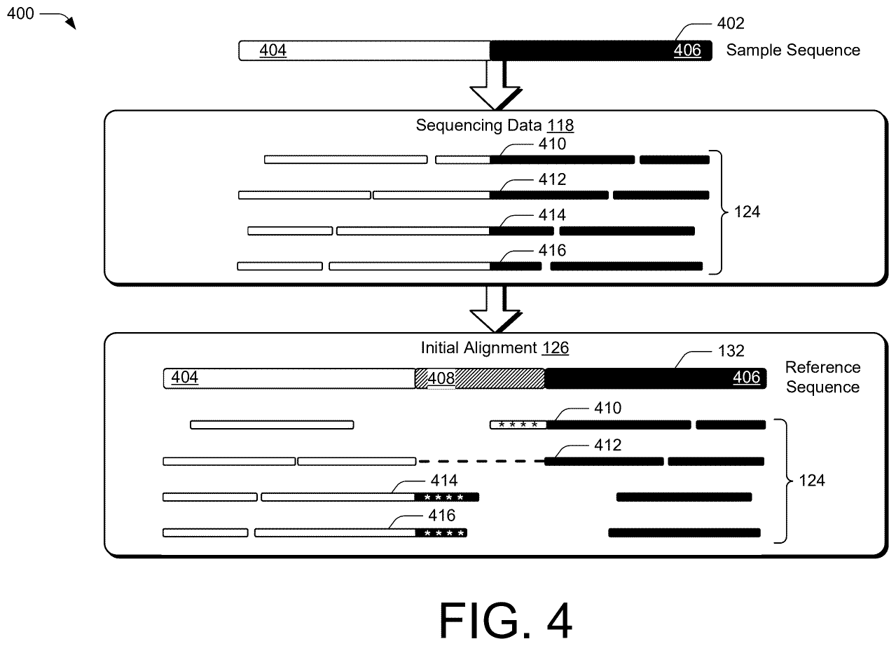

FIG. 4 depicts an overview 400 of alignment artifacts that may occur due to a deletion structural variant in a sample sequence. The overview 400 schematically depicts the sequencing data 118 generated for a sample sequence 402 (e.g., of a portion of the DNA 122), the sequencing data 118 comprising the sequencing reads 124.

The sample sequence 402 comprises a first genomic portion 404 (e.g., represented in white-fill/open-fill) and a second genomic portion 406 (e.g., represented in black-fill). However, the corresponding part of the reference sequence 132, shown in the initial alignment 126, further includes a third genomic portion 408 (e.g., represented in diagonal-fill) between the first genomic portion 404 and the second genomic portion 406, indicating that the sample sequence 402 has a deletion structural variant.

The sequencing reads 124 generated from the sample sequence 402 include a plurality of reads that extend from the first genomic portion 404 to the second genomic portion 406, including a first read 410, a second read 412, a third read 414, and a fourth read 416. These reads are shown as segments aligned with different parts of the sample sequence 402 in the sequencing data 118.

However, the initial alignment 126 includes alignment artifacts due to the at least one read alignment algorithm 130 (see FIG. 1) misaligning at least a portion of the sequencing reads 124 to preserve contiguity. In the present example, the first read 410, the second read 412, and the fourth read 416 are misaligned with the reference sequence 132. For instance, the portion of the first read 410 corresponding to the first genomic portion 404 is aligned to the third genomic portion 408 of the reference sequence 132, resulting in mismatches (e.g., indicated by asterisks). As another example, the portions of the third read 414 and the fourth read 416 corresponding to the second genomic portion 406 are aligned with the third genomic portion 408 of the reference sequence 132, which also results in mismatches. In contrast, the second read 412 is properly aligned as a split-read, with the first genomic portion 404 of the second read 412 aligned to the first genomic portion 404 of the reference sequence 132 and spaced apart from the second genomic portion 406 of the second read 412, which is properly aligned to the second genomic portion 406 of the reference sequence 132.

Accordingly, a deletion structural variant may not be identified from the initial alignment 126. Instead, smaller variants, such as single-nucleotide polymorphisms, may be identified at the mismatches. In the present example, these represent false positive variants.

Accordingly, the overview 400 demonstrates an example of alignment artifacts occurring during the process of aligning the sequencing reads 124 to the reference sequence 132. These alignment artifacts, e.g., corresponding to the alignment of the first read 410, third read 414, and fourth read 416, suppress the structural variant signal. However, due to the mismatches with the reference sequence 132, the first read 410, the third read 414, and the fourth read 416 may be detected as examples of the discordant reads 142, which may be analyzed by the techniques described herein to obtain structural variant information.

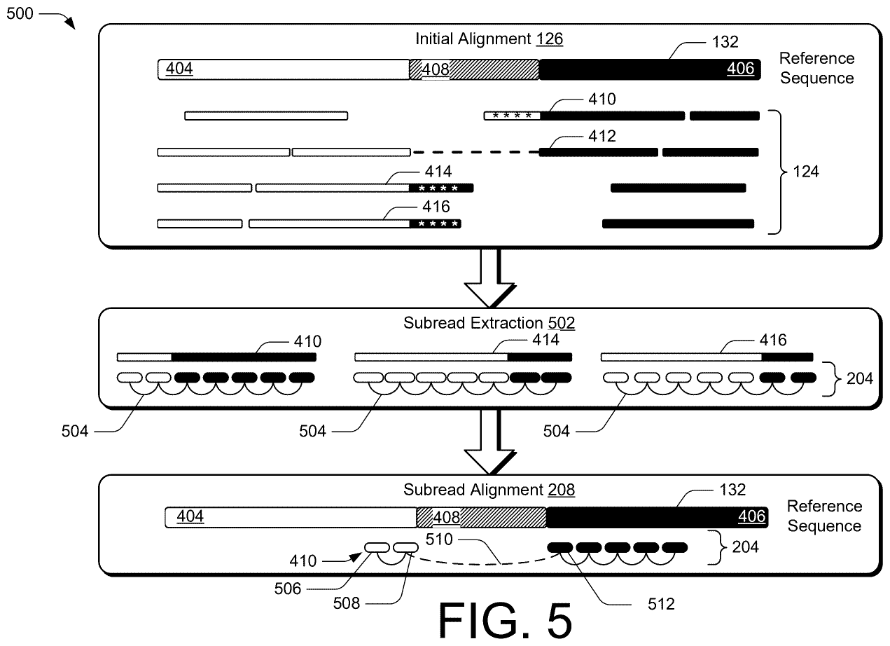

FIG. 5 depicts an overview 500 illustrating an example subread alignment corresponding to a deletion structural variant. By way of example, the overview 500 schematically depicts processes that may be performed by the subread analysis module 144 to generate the variant-indicative signal 148.

The overview 500 shows the initial alignment 126 depicted with respect to FIG. 4. A subread extraction 502 is performed on the discordant reads 142, e.g., the first read 410, the third read 414, and the fourth read 416. By way of example, the first read 410, the third read 414, and the fourth read 416 are each divided into shorter fragments, e.g., the subreads 204. Subread adjacencies 504 are represented as a series of connected arcs, illustrating the relationships between the subreads derived from a given original read.

The subread alignment 208 is generated, wherein the subreads 204 of a given discordant read 142 are aligned with the reference sequence 132. In the present example, a local alignment is performed. Moreover, for illustrative clarity, the subreads 204 for the first read 410 are shown, although it is to be appreciated that the subread alignment 208 also may be performed for the third read 414 and the fourth read 416. The subread alignment 208 is depicted below the reference sequence 132, showing how the individual subreads map to different portions of the reference. For example, a first subread 506 and a second subread 508 map to the first genomic portion 404 of the reference sequence 132, which results in no mismatches in the present example. This is in contrast to the initial alignment 126, where this portion of the first read 410 is misaligned to the third genomic portion 408. Moreover, the subread alignment 208 includes a third subread 512 correctly mapped to the second genomic portion 406 of the reference sequence 132.

The subread alignment 208 reveals a discordant junction 510 (e.g., one of the discordant junctions 212) between the second subread 508 and the third subread 512, where the alignment pattern of the subreads demonstrates discontinuity. For example, the second subread 508 and the third subread 512 are consecutive in the first read 410 but spaced apart on the reference sequence 132. The discordant junction 510 may indicate the presence of a structural variant. In this example, the discordant junction 510 corresponds to a deletion.

By comparing the sequencing reads 124 to the subread alignment 208, the subread analysis module 144 may identify signals (e.g., the discordant junctions 212) for potential structural variants that were obscured by alignment artifacts in the initial alignment 126. This approach may allow for more accurate detection of complex genomic rearrangements, particularly in cases where traditional alignment methods may struggle to correctly map reads spanning structural variant breakpoints.

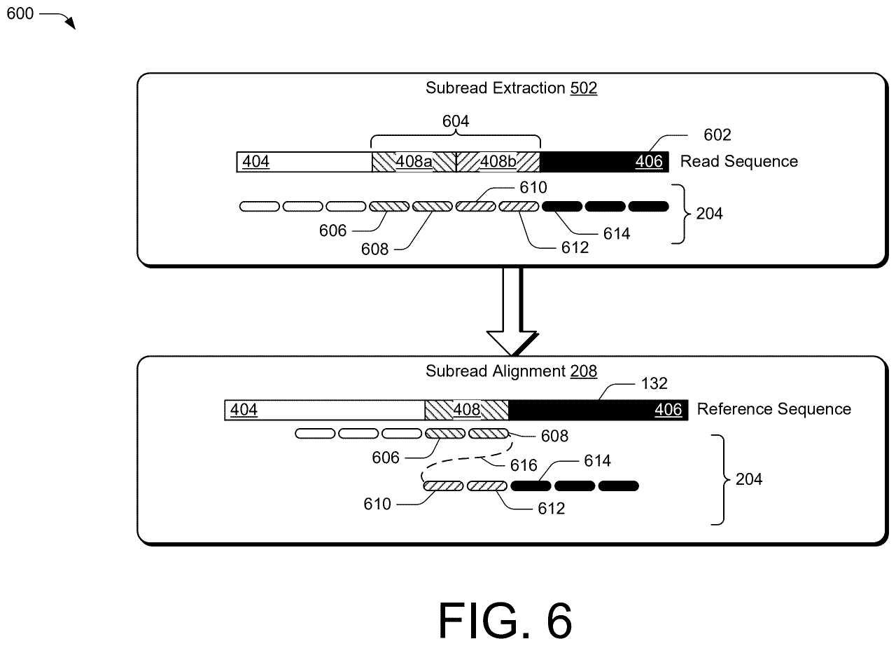

FIG. 6 depicts an overview 600 illustrating an example subread alignment corresponding to a duplication structural variant.

In the overview 600, the subread extraction 502 is performed (e.g., by the subread generator 202) on a read sequence 602 to generate the subreads 204. The read sequence 602 comprises the first genomic portion 404, the second genomic portion 406, and the third genomic portion 408. The third genomic portion 408 is duplicated, shown as a first duplicated portion 408a and a second duplicated portion 408b. As such, the read sequence 602 includes a duplication 604 as a structural variant. In the subread extraction 502, the read sequence 602 is divided into the subreads 204, including a first subread 606, a second subread 608, a third subread 610, a fourth subread 612, and a fifth subread 614.

The subread alignment 208 (e.g., as performed by the realignment algorithm 206) includes the subreads 204 aligned to the reference sequence 132. The reference sequence 132 includes the first genomic portion 404, the second genomic portion 406, and one copy of the third genomic portion 408 between the first genomic portion 404 and the second genomic portion 406, as in FIGS. 4 and 5. In the subread alignment 208 depicted in the overview 600, the subread alignment 208 includes a discordant junction 616 between the second subread 608 and the third subread 610. For example, the discordant junction 616 indicates subread alignment that is out of order compared to the read sequence 602, as the third subread 610 is consecutive with the second subread 608 in the read sequence 602 but not in the subread alignment 208. That is, the third subread 610 is aligned to the reference before (e.g., upstream of) the second subread 608 in the subread alignment 208 (e.g., the order of the second subread 608 and the third subread 610 is flipped in the subread alignment 208). The discordant junction 616 is an example of the discordant junctions 212 and provides a signal that can be used to identify and characterize the duplication 604 in the read sequence 602.

As such, the overview 600 demonstrates how the subread analysis module 144 may be used to identify the duplication 604 by breaking down the longer read sequence 602 into the smaller subreads 204 (e.g., using the subread generator 202) and analyzing their alignment patterns (e.g., using the realignment algorithm 206) against the reference sequence 132 to identify the discordant junctions 212, which may be used by the LBG analysis module 146 to identify latent breakpoints and/or the variant calling module 150 to identify structural variants. In contrast, a traditional alignment (e.g., performed using the at least one read alignment algorithm 130) may indicate the second duplicated portion 408b is a novel insertion rather than indicating the duplication 604.

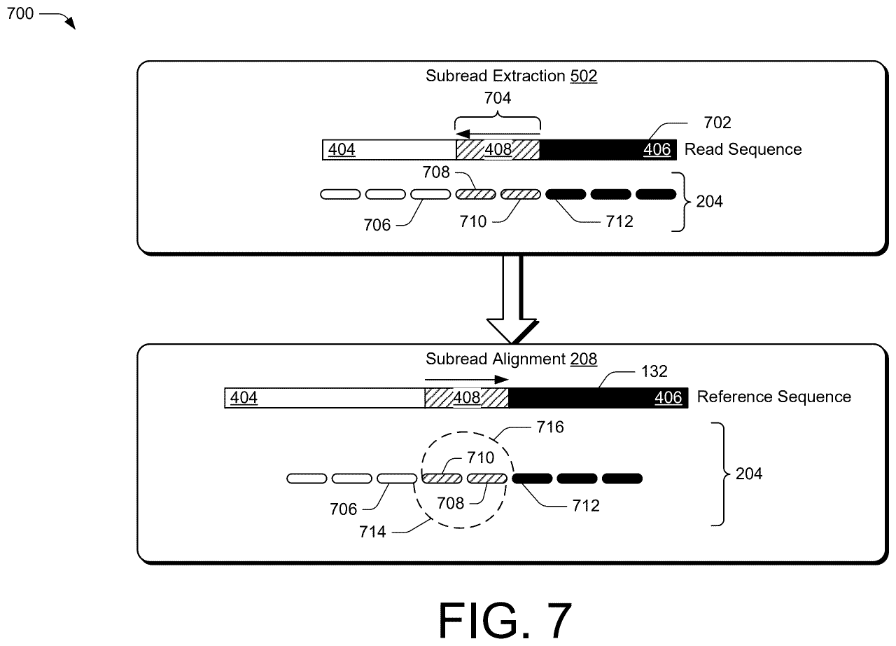

FIG. 7 depicts an overview 700 illustrating an example subread alignment corresponding to an inversion structural variant.

In the overview 700, the subread extraction 502 is performed (e.g., by the subread generator 202) on a read sequence 702 to generate the subreads 204. The read sequence 702 comprises the first genomic portion 404, the second genomic portion 406, and the third genomic portion 408. The third genomic portion 408 is inverted, shown as an arrow in FIG. 7. As such, the read sequence 702 includes an inversion 704 as a structural variant.

In the subread extraction 502, the read sequence 702 is divided into the subreads 204, including a first subread 706, a second subread 708, a third subread 710, and a fourth subread 712. The subread alignment 208 (e.g., as performed by the realignment algorithm 206) includes the subreads 204 aligned to the reference sequence 132. The reference sequence 132 includes the first genomic portion 404, the second genomic portion 406, and the third genomic portion 408 between the first genomic portion 404 and the second genomic portion 406, as in FIGS. 4-6. The third genomic portion 408 is oriented in an opposite direction as compared to the read sequence 702, as indicated by the arrow above the third genomic portion 408. In the subread alignment 208 depicted in the overview 700, the subread alignment 208 includes a first discordant junction 714 between the first subread 706 and the second subread 708, and a second discordant junction 716 between the third subread 710 and the fourth subread 712. For example, the first discordant junction 714 indicates the second subread 708 is mapped to a different strand of the reference sequence 132 compared to the first subread 706 in the subread alignment 208, despite originating from the same strand in the read sequence 702. As another example, the second discordant junction 716 indicates another subread alignment to an unexpected location, as the third subread 710 is contiguous with the fourth subread 712 and on the same strand in the read sequence 702, but the third subread 710 maps to a different strand than the fourth subread 712 in the subreads 204. The first discordant junction 714 and the second discordant junction 716 are examples of the discordant junctions 212 and provide a signal that can be used to identify and characterize the inversion 704 in the read sequence 702.

As such, the overview 700 demonstrates how the subread analysis module 144 may be used to identify the inversion 704 by breaking down the longer read sequence 702 into the smaller subreads 204 (e.g., using the subread generator 202) and analyzing their alignment patterns (e.g., using the realignment algorithm 206) against the reference sequence 132 to identify the discordant junctions 212, which may be used by the LBG analysis module 146 to identify latent breakpoints and/or the variant calling module 150 to identify structural variants. In contrast, a traditional alignment (e.g., performed using the at least one read alignment algorithm 130) may report the inversion 704 as a novel insertion and deletion or a mismatched sequence.

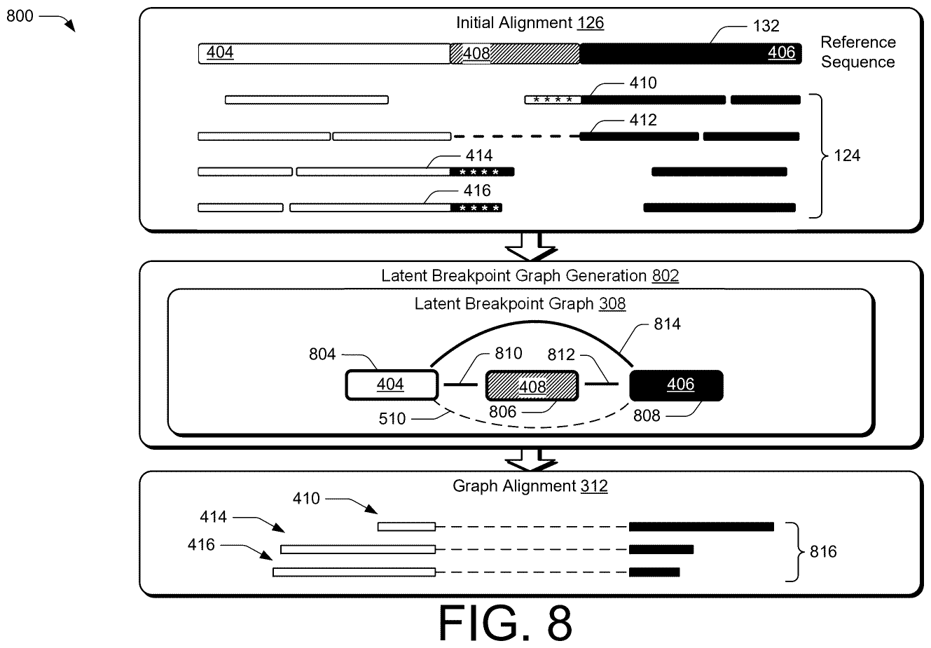

FIG. 8 depicts an overview 800 illustrating an example latent breakpoint graph analysis.

The overview 800 shows the initial alignment 126 introduced with respect to FIG. 4. For example, the initial alignment 126 includes the reference sequence 132 comprising the first genomic portion 404, the third genomic portion 408, and the second genomic portion 406. The sequencing reads 124 are shown mapped to the reference sequence 132, including the first read 410, the second read 412, the third read 414, and the fourth read 416 (e.g., of the sample sequence 402 of FIG. 4). The initial alignment 126 includes alignment artifacts, as previously discussed with respect to FIG. 4.

In the overview 800, a latent breakpoint graph generation 802 is performed based on the discordant reads 142 of the initial alignment 126, resulting in the latent breakpoint graph 308. The latent breakpoint graph 308 comprises a first node 804 representing the first genomic portion 404, a second node 806 representing the third genomic portion 408, and a third node 808 representing the second genomic portion 406. The nodes are connected by edges, including a first edge 810 between the first node 804 and the second node 806, a second edge 812 between the second node 806 and the third node 808, and a latent edge 814 directly connecting the first node 804 and the third node 808. The first edge 810 and the second edge 812 correspond to reference adjacencies (e.g., as determined from the reference sequence 132), whereas the latent edge 814 corresponds to the discordant junction 510 introduced with respect to FIG. 5. Accordingly, the latent edge 814 may be determined from the discordant junctions 212 (e.g., the discordant junction 510). Alternatively, or in addition, the latent edge 814 may be inferred from the split-read alignment of the second read 412 if an initial alignment is provided.

The graph alignment 312 is generated (e.g., by the graph alignment algorithm 310) based on the latent breakpoint graph 308. In the graph alignment 312, reads can map contiguously across the latent edge 814 without incurring an alignment penalty. The graph alignment 312 shows the alignment of the sequencing reads 124 to the latent breakpoint graph 308. When the graph alignment 312 is converted to a standard reference alignment, the first read 410, the third read 414, and the fourth read 416 are depicted as split-reads 816, indicating a deletion. The split-reads 816 are represented by dashed lines connecting different parts of the reads.

As such, the overview 800 demonstrates how the LBG analysis module 146 may be used to identify structural variants by first generating the latent breakpoint graph 308 based on the discordant reads 142 in the initial alignment 126 and/or the discordant junctions 212 (e.g., novel junctions identified by the subread analysis module 144 directly from the sequencing reads 124 or using the initial alignment 126), and then realigning the sequencing reads 124 to the latent breakpoint graph 308 to reveal potential variants through the latent edges denoting novel adjacencies. Whether alone or in combination with the subread analysis performed by the subread analysis module 144, this approach may allow for more accurate detection of complex genomic rearrangements by capturing relationships between genomic regions that are not apparent in the reference sequence 132, even when the reference sequence 132 is a pangenome.

Having discussed example details of the techniques for variant identification in the presence of alignment artifacts, consider now example procedures to illustrate additional aspects of the techniques.

### Example Procedures

This section describes procedures for variant identification in the presence of alignment artifacts in one or more implementations. Aspects of the procedures may be implemented in hardware, firmware, or software, or a combination thereof. The procedures are shown as a set of blocks that specify operations performed by one or more devices and are not necessarily limited to the orders shown for performing the operations by the respective blocks. In at least some implementations, at least a portion of the procedures are performed by a suitably configured device, such as the sequencing data processor 108 of FIG. 1, by executing instructions stored in a non-transitory computer-readable storage medium.

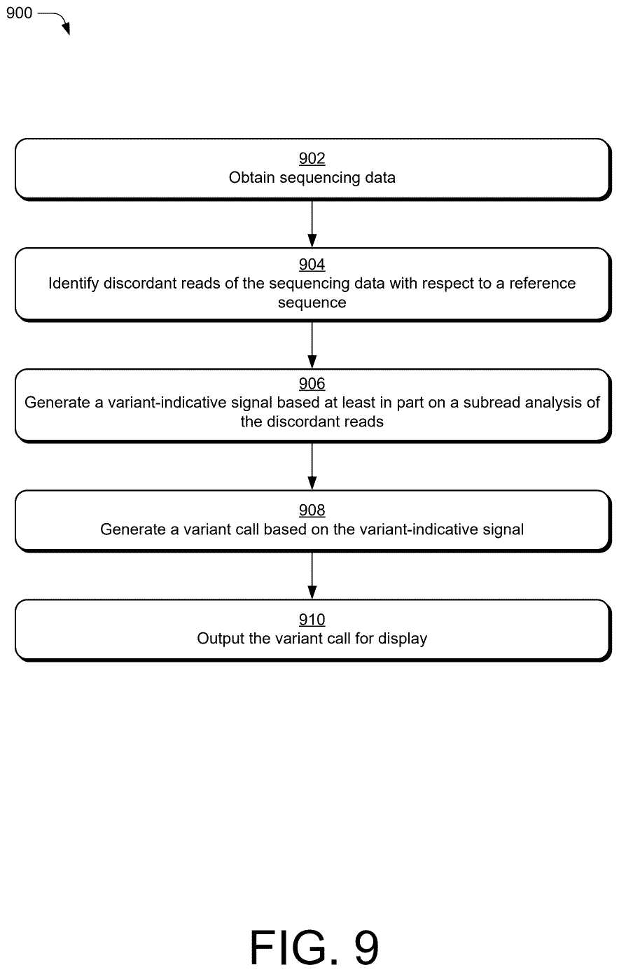

FIG. 9 depicts an example procedure 900 in which structural variant identification is performed.

Sequencing data is obtained (block 902). By way of example, the sequencing data may be generated by a DNA sequencer 106 using a long-read or a short-read sequencing technique. The DNA sequencer 106 may produce the sequencing data 118 from DNA 122 obtained from the biological sample 120, for instance. The sequencing data 118 may include long reads typically ranging from approximately 2000 bases to 1,000,000 bases and/or short reads typically ranging from approximately 10 bases to approximately 1000 bases.

Discordant reads of the sequencing data are identified with respect to a reference sequence (block 904). By way of example, reads that map discordantly to the reference sequence 132, referred to herein as the discordant reads 142, may be identified based on the alignment characteristics from a full or partial alignment of the sequencing reads 124 to the reference sequence 132 (e.g., the initial alignment 126) and/or using a k-mer analysis. The partial alignment and the k-mer analysis may be faster and less computing resource intensive than the full alignment. In at least one implementation, all reads may be considered discordant and passed to the subread analysis module 144, which may be more resource intensive but may result in higher sensitivity.

In at least one implementation where a full alignment is generated, the alignment module 128 may generate the initial alignment 126 using the at least one read alignment algorithm 130, which may map the sequencing reads 124 to the reference sequence 132. The reference sequence 132 may be a linear reference sequence or a pangenome, for example. Subsequently, the discordant read analysis module 136 may analyze the initial alignment 126 to detect reads exhibiting the alignment characteristics of improper mapping, such as large insertions/deletions, clusters of small insertions/deletions, and/or stretches of mismatches, using the discordant read identification algorithm 138 and the parameters 140. These discordant reads 142 may encode structural variants and exhibit poor mapping due to alignment artifacts caused by the read alignment algorithm 130, which may present as false positive SNPs.

In at least one variation where a partial alignment is generated, the alignment module 128 (or the discordant read identification algorithm 138, such as when the alignment module 128 is not configured to perform the partial alignment) may map beginning and end portions of the sequencing reads 124 to the reference sequence 132. Because a distance between the beginning portion and the end portion of a read is known, the discordant read analysis module 136 may identify the discordant reads 142 as those where the distance differs when mapped to the reference sequence 132 by at least a threshold (e.g., as defined by the parameters 140).

In at least one other variation, the discordant read identification algorithm 138 may analyze the sequencing reads 124 using the k-mer analysis. The k-mer analysis may include dividing the sequencing reads 124 into tokens of k length in an overlapping or non-overlapping fashion. The tokens are then mapped to the reference sequence 132. The discordant reads 142 include those having tokens that map in the wrong order and/or to both strands of the reference sequence 132.

In at least one implementation, the discordant reads 142 identified using the partial alignment and/or the k-mer analysis are verified using the full alignment analysis. For instance, the candidate discordant reads may be fully aligned to the reference sequence 132 (e.g., via the at least one read alignment algorithm 130) and analyzed by the discordant read analysis module 136 to detect reads exhibiting signs of improper mapping, as described above. In at least one implementation, the discordant reads 142 are directly processed using subread analysis (e.g., performed by the subread analysis module 144) without generating an alignment.

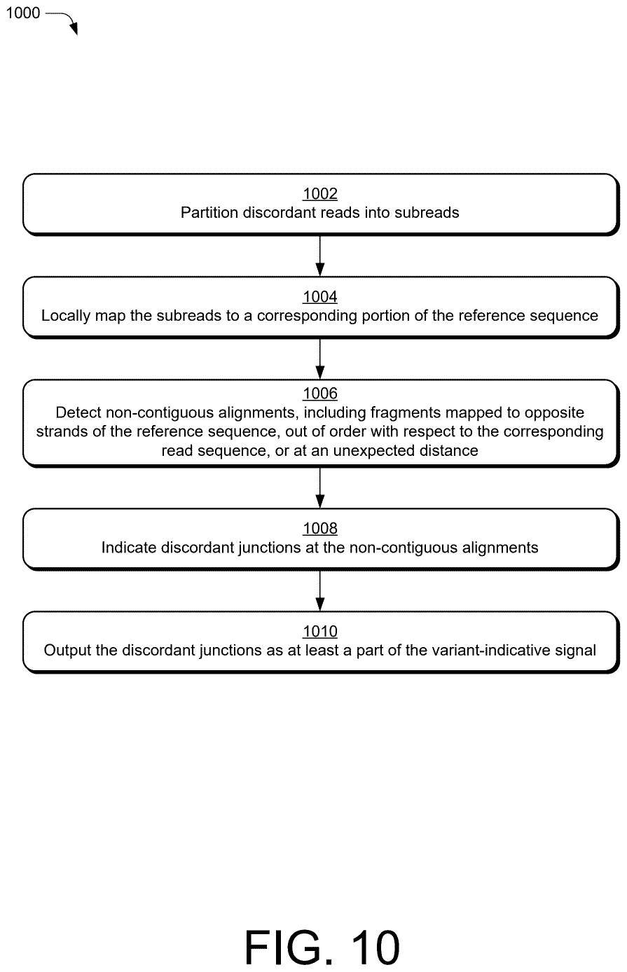

A variant-indicative signal is generated based at least in part on a subread analysis of the discordant reads (block 906). By way of example, the subread analysis may include partitioning the discordant reads 142 into shorter fragments called subreads, locally mapping (or re-mapping) these subreads to corresponding portions of the reference sequence 132, and detecting non-contiguous alignments (such as fragments mapped to opposite strands or out of order), as will be further described with respect to FIG. 10 and explained above with respect to FIGS. 1, 2, and 5-7. As such, the variant-indicative signal 148 may include the discordant junctions 212.

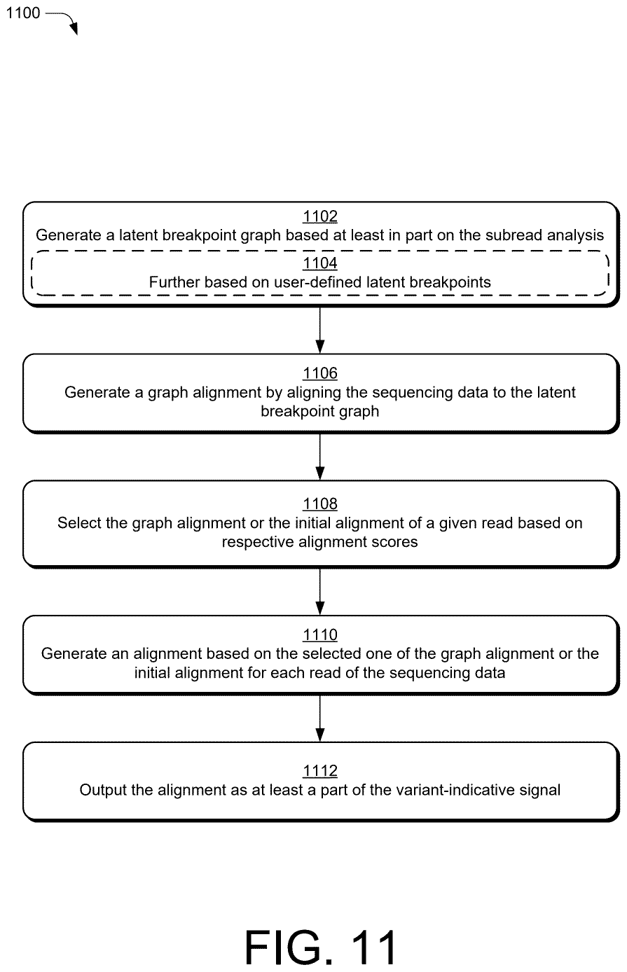

In at least one implementation, additionally or optionally, a latent breakpoint graph is generated based on the subread analysis. The latent breakpoint graph 308 may be a graph representation of potential genomic rearrangements based on the novel adjacencies discovered from (e.g., indicated by) the discordant reads 142. The sequencing reads 124 may be aligned to the latent breakpoint graph 308 to generate an alignment (e.g., the corrected alignment 314), as will be further described with respect to FIG. 11. In such instances, the variant-indicative signal 148 may include the corrected alignment 314. It is to be appreciated that the corrected alignment 314 may enable single nucleotide polymorphisms (SNPs) and short insertions or deletions (indels) to be more accurately identified in addition to more accurate structural variant identification.

The variant-indicative signal 148 generated through the subread analysis (and, additionally or optionally, the latent breakpoint graph analysis) may capture complex genomic rearrangements that may be at least partially suppressed in the initial alignment 126, which may improve the accuracy and sensitivity of structural variant detection.

A variant call is generated based on the variant-indicative signal (block 908). By way of example, the variant calling module 150 may use one or more variant calling algorithms 152 to generate the variant call 154 based at least in part on the variant-indicative signal 148. The variant call 154 may include an indication of structural variants that are determined to be present in the sequencing reads 124 compared to the reference sequence 132. The variant call 154 may further include information about small variants, such as SNPs and indels.

In at least one implementation, the variant calling module 150 may analyze both the initial alignment 126 and the variant-indicative signal 148 to determine the presence and characteristics of variants. In at least one variation, the variant calling module 150 may analyze the corrected alignment 314 and not the initial alignment 126. In at least one variation, the variant calling module 150 may analyze only the novel junction information directly provided by the subread analysis module 144 (e.g., via the discordant junctions 212 output as the variant-indicative signal 148). In some instances, the variant calling module 150 may employ statistical methods to assess the likelihood of each potential variant, considering factors such as sequencing depth, mapping quality, and allele frequencies. The resulting variant call 154 may provide a comprehensive summary of the genomic differences detected between the sequencing data 118 and the reference sequence 132, including both large structural variations and smaller sequence-level changes.

The variant call is output for display (block 910). By way of example, the variant call 154 may be displayed on the display device 156 of the client device 104, allowing users to visualize and analyze the identified structural variants.

In this way, the procedure 900 may provide an approach for identifying variants in the sequencing data 118 in the presence of alignment artifacts. By first identifying the discordant reads 142 and then further analyzing the discordant reads 142 via the subread analysis, the procedure 900 may extract signals (e.g., the discordant junctions 212) from potential structural variants that might be missed by traditional alignment methods alone. By generating the variant-indicative signal 148 using the subread analysis module 144 and/or the LBG analysis module 146, complex genomic rearrangements, including inversions, translocations, and large insertions or deletions, may be identified. Moreover, an accuracy of a short variant call may be increased. The procedure 900 may also provide flexibility in variant calling by utilizing both the initial alignment 126 and the variant-indicative signal 148.

FIG. 10 depicts an example procedure 1000 for generating a variant-indicative signal using a subread analysis. In at least one implementation, the procedure 1000 is a sub-routine of the procedure 900 of FIG. 9 (e.g., at the block 906).

The discordant reads are partitioned into subreads (block 1002). By way of example, the subread generator 202 may divide a given discordant read 142 into subreads 204 of a configurable length, typically ranging from 50 to 1000 base pairs. This partitioning may allow for more precise local alignments with the reference sequence 132 compared to the corresponding full-length read sequence, particularly in the case of long-read sequencing data. In at least one variation, each of the sequencing reads 124 is partitioned into subreads and analyzed by the subread analysis module 144 without first evaluating for discordance with respect to the reference sequence 132.

The subreads are locally mapped to a corresponding portion of the reference sequence (block 1004). By way of example, the realignment algorithm 206 may perform a more precise local alignment of the subreads 204 to the reference sequence 132, generating the subread alignment 208. This may allow for a more detailed analysis of the read alignment, particularly in regions where the reference sequence 132 and the read sequence differ due to a structural rearrangement. In at least one variation, the subreads may be mapped to the entire reference.

Non-contiguous alignments are detected, including fragments mapped to opposite strands of the reference sequence, out of order with respect to the corresponding read sequence, or at an unexpected distance (block 1006). By way of example, the discordant junction identifier 210 may scan the subread alignment 208 to identify novel adjacencies that form the discordant junctions 212. These discordant junctions 212 may serve as indicators of structural variants, e.g., deletions, inversions, translocations, and/or duplications that may be missed or mischaracterized by the read alignment algorithm 130. By way of example, the non-contiguous alignments may include subreads mapping to the reference sequence 132 at a distance from each other that is different than their distance within the read (e.g., an unexpected distance based on the read, which is indicative of a potential deletion), subreads mapping out of order (indicative of a potential duplication), and/or subreads mapping to different strands of the reference sequence 132 (indicative of a potential inversion). An unexpected distance may also be referred to herein as a discordant distance because the distance is not in agreement with the structure of the read.

Discordant junctions are indicated at the non-contiguous alignments (block 1008). By way of example, the discordant junction identifier 210 may indicate the locations where non-contiguous alignments occur as well as a type of novel adjacency (e.g., opposite strand, out of order, unexpected distance). These discordant junctions 212 may indicate potential breakpoints or structural variations in the read sequence compared to the reference sequence 132.

The discordant junctions are output as at least a part of the variant-indicative signal (block 1010). By way of example, the discordant junctions 212 may be included in the variant-indicative signal 148, which may be further analyzed by the variant calling module 150 to generate the variant call 154, as elaborated with respect to FIGS. 1 and 9. In at least one variation, the discordant junctions 212 are used to construct the latent breakpoint graph 308 for generating the corrected alignment 314, as further described below with respect to FIG. 11.

FIG. 11 depicts an example procedure 1100 for generating a variant-indicative signal using a latent breakpoint graph analysis. In at least one implementation, the procedure 1100 is a sub-routine of the procedure 900 of FIG. 9 (e.g., at the block 906).

A latent breakpoint graph is generated based at least in part on the subread analysis (block 1102). By way of example, the graph constructor 306 may generate the latent breakpoint graph 308 based on the discordant junctions 212. The latent breakpoint graph 308 may represent a personalized representation of the genomic structure of the biological sample 120, capturing potential structural variations and rearrangements. For example, the discordant reads 142 may indicate an area of mismatch without indicating a deletion (or other structural variant) is present, and the discordant junctions 212 may indicate the deletion (or other structural variant) rather than the area of mismatch.

In one or more implementations, additionally or optionally, the latent breakpoint graph is further based on user-defined latent breakpoints (block 1104). By way of example, the graph constructor 306 may incorporate the user-defined input(s) 304 for potential or suspected adjacencies that are not present in the reference sequence 132, allowing for customization of the analysis process based on specific research objectives or known genomic characteristics of the sample.

A graph alignment is generated by aligning the sequencing data to the latent breakpoint graph (block 1106). By way of example, the graph alignment algorithm 310 may align the sequencing reads 124 to the latent breakpoint graph 308, generating the graph alignment 312. This alignment process may reveal alignments that better explain sequencing data 118 by, for example, reducing mismatches or other alignment artifacts.

The graph alignment or the initial alignment for a given read is selected based on respective alignment scores (block 1108). By way of example, the graph alignment algorithm 310 may compare the respective alignment scores of the sequencing reads 124 in the graph alignment 312 with corresponding scores from the initial alignment 126 in order to determine which alignment (e.g., the initial alignment 126 or the graph alignment 312) better fits the observed sequencing data 118. An alignment score, for example, may take into account factors such as the number of matching bases, gaps, insertions, and deletions. An initial alignment score for the initial alignment 126, for example, may reflect how well the read maps to the reference sequence 132. A graph alignment score for the graph alignment 312, on the other hand, may indicate how accurately the read follows paths through the latent breakpoint graph 308, including any novel adjacencies represented by latent edges.

In at least one implementation, the graph alignment algorithm 310 may assign positive values to matches between the read and the reference sequence 132 or graph alignment 312 and negative values to mismatches, gaps, insertions, and/or deletions. By way of example, a split-read alignment and no or few mismatches may have a larger score than a contiguous read alignment (e.g., no gap) with a greater number of mismatches. In this context, a higher score denotes a more accurate alignment. In at least one variation, however, a different scoring system may be used than that described above. By comparing these scores, the graph alignment algorithm 310 may determine which alignment more accurately represents the true genomic structure of the DNA 122 from the biological sample 120.

An alignment is generated based on the selected one of the graph alignment or the initial alignment for each read of the sequencing data (block 1110). By way of example, the alignment with the better (e.g., higher) score for a given sequencing read 124 may be selected for inclusion in the alignment (e.g., the corrected alignment 314). Accordingly, the corrected alignment 314 may be more accurate than the initial alignment 126. It is to be appreciated that when the initial alignment 126 is not performed or is a partial alignment, the alignment comprises the graph alignment 312.

The alignment is output as at least a part of the variant-indicative signal (block 1112). By way of example, the corrected alignment 314 may be included in the variant-indicative signal 148, which can be further analyzed by the variant calling module 150 to generate the variant call 154, as elaborated with respect to FIGS. 1 and 9.

Having described the example procedures in accordance with one or more implementations, consider now an example system and device that can be utilized to implement the various techniques described herein.

### Example System and Device

FIG. 12 illustrates an example system generally at 1200 that includes an example computing device 1202 that is representative of one or more computing systems and/or devices that may implement the various techniques described herein. This is illustrated through inclusion of the sequencing data processor 108. The computing device 1202 may be, for example, a server of a service provider, a device associated with a client (e.g., a client device), an on-chip system, and/or any other suitable computing device or computing system.

The example computing device 1202 as illustrated includes a processing system 1204, one or more computer-readable media 1206, and one or more I/O interfaces 1208 that are communicatively coupled, one to another. Although not shown, the computing device 1202 may further include a system bus or other data and command transfer system that couples the various components, one to another. A system bus can include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 1204 is representative of functionality to perform one or more operations using hardware. Accordingly, the processing system 1204 is illustrated as including hardware elements 1210 that may be configured as processors, functional blocks, and so forth. This may include implementation in hardware as an application-specific integrated circuit or other logic device formed using one or more semiconductors. The hardware elements 1210 are not limited by the materials from which they are formed or the processing mechanisms employed therein. For example, processors may be comprised of semiconductor(s) and/or transistors (e.g., electronic integrated circuits (ICs)). In such a context, processor-executable instructions may be electronically executable instructions.

The computer-readable storage media 1206 is illustrated as including memory/storage 1212 thereon. The memory/storage 1212 represents memory/storage capacity associated with one or more computer-readable media. The memory/storage 1212 may include volatile media (such as random-access memory (RAM)) and/or nonvolatile media (such as read-only memory (ROM), Flash memory, optical disks, magnetic disks, and so forth). The memory/storage 1212 may include fixed media (e.g., RAM, ROM, a fixed hard drive, and so on) as well as removable media (e.g., flash memory, a removable hard drive, an optical disc, and so forth). The computer-readable media 1206 may be configured in a variety of other ways as further described below.

Input/output interface(s) 1208 are representative of functionality to allow a user to enter commands and information to the computing device 1202, and also allow information to be presented to the user and/or other components or devices using various input/output devices. Examples of input devices include a keyboard, a cursor control device (e.g., a mouse), a microphone, a scanner, touch functionality (e.g., capacitive or other sensors that are configured to detect physical touch), a camera (e.g., which may employ visible or non-visible wavelengths such as infrared frequencies to recognize movement as gestures that do not involve touch), and so forth. Examples of output devices include a display device (e.g., a monitor or projector), speakers, a printer, a network card, a tactile-response device, and so forth. Thus, the computing device 1202 may be configured in a variety of ways as further described below to support user interaction.

Various techniques may be described herein in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. The features of the techniques described herein are platform-independent, meaning that the techniques may be implemented on a variety of commercial computing platforms having a variety of processors.

For instance, the terms “module,” “functionality,” and “component” may include a hardware and/or software system that operates to perform one or more functions. For example, a module, functionality, or component may include a computer processor, a controller, or another logic-based device that performs operations based on instructions stored on a tangible and non-transitory computer-readable storage medium, such as a computer memory. Alternatively, a module, functionality, or component may include a hard-wired device that performs operations based on hard-wired logic of the device. Various modules, systems, and components shown in the attached figures may represent the hardware that operates based on software or hardwired instructions, the software that directs hardware to perform the operations, or a combination thereof.

An implementation of the described modules and techniques may be stored on or transmitted across some form of computer-readable media. The computer-readable media may include a variety of media that may be accessed by the computing device 1202. By way of example, and not limitation, computer-readable media may include “computer-readable storage media” and “computer-readable signal media.”

“Computer-readable storage media” may refer to media and/or devices that enable persistent and/or non-transitory storage of information in contrast to mere signal transmission, carrier waves, or signals per se. Thus, the term “computer-readable storage media” refers to non-signal bearing media. The computer-readable storage media include hardware such as volatile and non-volatile, removable and non-removable media, and/or storage devices implemented in a method or technology suitable for storage of information such as computer readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage devices, tangible media, or articles of manufacture suitable to store the desired information and which may be accessed by a computer.

“Computer-readable signal media” may refer to a signal-bearing medium that is configured to transmit instructions to the hardware of the computing device 1202, such as via a network. Signal media typically may embody computer-readable instructions, data structures, program modules, or other data in a modulated data signal, such as carrier waves, data signals, or other transport mechanisms. Signal media also include any information delivery media. The term “modulated data signal” means a signal that has one or more of its characteristics set or changed in such a manner as to encode information in the signal. By way of example, and not limitation, communication media include wired media such as a wired network or direct-wired connection, and wireless media such as acoustic, RF, infrared, and other wireless media.

As previously described, hardware elements 1210 and computer-readable media 1206 are representative of modules, programmable device logic and/or fixed device logic implemented in a hardware form that may be employed in some examples to implement at least some aspects of the techniques described herein, such as to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as a hardware utilized to store instructions for execution, e.g., the computer-readable storage media described previously.

Combinations of the foregoing may also be employed to implement various techniques described herein. Accordingly, software, hardware, or executable modules may be implemented as one or more instructions and/or logic embodied on some form of computer-readable storage media and/or by one or more hardware elements 1210. The computing device 1202 may be configured to implement particular instructions and/or functions corresponding to the software and/or hardware modules. Accordingly, implementation of a module that is executable by the computing device 1202 as software may be achieved at least partially in hardware, e.g., through use of computer-readable storage media and/or hardware elements 1210 of the processing system 1204. The instructions and/or functions may be executable/operable by one or more articles of manufacture (for example, one or more computing devices 1202 and/or processing systems 1204) to implement techniques, modules, and examples described herein.

The techniques described herein may be supported by various configurations of the computing device 1202 and are not limited to the specific examples of the techniques described herein. This functionality may also be implemented all or in part through use of a distributed system, such as over a “cloud” 1214 via a platform 1216 as described below.

The cloud 1214 includes and/or is representative of a platform 1216 for resources 1218, which are depicted as including the sequencing data processor 108. The platform 1216 abstracts the underlying functionality of hardware (e.g., servers) and software resources of the cloud 1214. The resources 1218 may include applications and/or data that can be utilized while computer processing is executed on servers that are remote from the computing device 1202. Resources 1218 can also include services provided over the Internet and/or through a subscriber network, such as a cellular or Wi-Fi network.

The platform 1216 may abstract resources and functions to connect the computing device 1202 with other computing devices. The platform 1216 may also serve to abstract scaling of resources to provide a corresponding level of scale to meet the encountered demand for the resources 1218 that are implemented via the platform 1216. Accordingly, in an interconnected device example, implementation of functionality described herein may be distributed throughout the system 1200. For example, the functionality may be implemented in part on the computing device 1202 as well as via the platform 1216 that abstracts the functionality of the cloud 1214.

### Conclusion

Although the invention has been described in language specific to structural features and/or methodological acts, it is to be understood that the invention defined in the appended claims is not necessarily limited to the specific features or acts described. Rather, the specific features and acts are disclosed as example forms of implementing the claimed invention.

What is claimed is:

1. A method for structural variant identification, comprising:
identifying discordant reads of sequencing data with respect to a reference sequence;
generating a variant-indicative signal based at least in part on a subread analysis of the discordant reads, the variant-indicative signal including one or both of a discordant junction signal or an alignment generated using a latent breakpoint graph; and
generating a variant call based at least in part on the variant-indicative signal, the variant call identifying a structural variant in the sequencing data as compared to the reference sequence.

2. The method of claim 1, wherein generating the variant-indicative signal based at least in part on the subread analysis of the discordant reads comprises:
partitioning the discordant reads into subreads;
mapping the subreads to a corresponding portion of the reference sequence;
detecting novel adjacencies between at least a portion of consecutive subread pairs of the subreads; and
outputting the novel adjacencies as the discordant junction signal.

3. The method of claim 2, wherein the novel adjacencies comprise a junction between a first subread and a second subread that map to locations on the reference sequence with a different relative distance compared to their respective positions in a corresponding read.

4. The method of claim 2, wherein the novel adjacencies comprise a junction between a first subread and a second subread that map to the reference sequence in a different order as compared to a corresponding read.

5. The method of claim 2, wherein the novel adjacencies comprise a junction between a first subread and a second subread that map to opposite strands of the reference sequence, while originating from a same strand in a corresponding read.

6. The method of claim 1, wherein generating the variant-indicative signal comprises:
generating the latent breakpoint graph based at least in part on the discordant junction signal, the latent breakpoint graph including nodes representing genomic segments from the reference sequence and edges connecting the nodes, wherein:
a first set of edges represents adjacencies between the genomic segments in the reference sequence; and
a second set of edges represents novel adjacencies defined based on the discordant junction signal; and
generating a graph alignment by aligning the sequencing data to the latent breakpoint graph.

7. The method of claim 6, further comprising:
receiving an initial alignment of the sequencing data mapped to the reference sequence;
for each read of the sequencing data, selecting between the graph alignment and the initial alignment based on respective alignment scores; and
generating the alignment by using a selected one of the graph alignment or the initial alignment for each read of the sequencing data.

8. The method of claim 6, wherein generating the latent breakpoint graph is further based on user input, and wherein the user input defines a third set of edges between the nodes.

9. The method of claim 1, wherein identifying the discordant reads of the sequencing data is based on an initial alignment of the sequencing data to the reference sequence, and wherein the initial alignment is a full alignment of the sequencing data to the reference sequence or a partial alignment of the sequencing data to the reference sequence.

10. The method of claim 1, wherein identifying the discordant reads of the sequencing data comprises performing a k-mer analysis by:
dividing reads of the sequencing data into tokens;
mapping the tokens to the reference sequence; and
indicating a read of the sequencing data as discordant in response to the tokens of the read mapping to multiple strands of the reference sequence or at a discordant distance.

11. A system for structural variant identification, comprising:
a processing system; and
a computer-readable storage medium having instructions stored thereon that, when executed by the processing system, cause the processing system to perform operations comprising:
identifying discordant reads of sequencing data obtained for a sample, the discordant reads comprising reads of the sequencing data that include at least one of a threshold number of mismatches to a reference sequence, an insertion with respect to the reference sequence, a deletion with respect to the reference sequence, an inversion with respect to the reference sequence, or a split alignment;
generating a variant-indicative signal based at least in part on a subread analysis of the discordant reads, the subread analysis defining novel adjacencies in the discordant reads; and
generating a variant call of the sample based at least in part on the variant-indicative signal, the variant call identifying a structural variant in the sequencing data as compared to the reference sequence.

12. The system of claim 11, wherein generating the variant-indicative signal based at least in part on the subread analysis of the discordant reads comprises:
partitioning the discordant reads into subreads;
mapping the subreads to a corresponding portion of the reference sequence; and
detecting the novel adjacencies between at least a portion of the subreads based on at least one of:
a change in a relative position of the subreads as compared to their positions in the discordant reads;
a change in an order of the subreads as compared to their order in the discordant reads; or
an alignment of the subreads to different strands of the reference sequence.

13. The system of claim 11, wherein generating the variant-indicative signal comprises:
generating a latent breakpoint graph based at least in part on the novel adjacencies by:
defining nodes representing genomic segments from the reference sequence;
defining a first set of edges between the nodes representing adjacencies between the genomic segments in the reference sequence; and
defining a second set of edges between the nodes representing the novel adjacencies; and
generating a graph alignment by aligning the sequencing data to the latent breakpoint graph.

14. The system of claim 13, wherein the operations further comprise:
generating an initial alignment by mapping the sequencing data to the reference sequence; and
generating an updated alignment based on the graph alignment and the initial alignment by:
for each read of the sequencing data, selecting one of the graph alignment or the initial alignment based on respective alignment scores; and
including the selected one of the graph alignment or the initial alignment in the updated alignment.

15. The system of claim 13, wherein the latent breakpoint graph further comprises:
a third set of edges between the genomic segments in the reference sequence defined by user input.

16. A method for structural variant identification, comprising:
obtaining sequencing data of a sample;
generating a variant-indicative signal based on a subread analysis of sequencing reads of the sequencing data, the subread analysis defining novel adjacencies in the sequencing reads;
and generating a variant call based at least in part on the variant-indicative signal and a reference sequence, the variant call identifying a structural variant in the sequencing data as compared to the reference sequence.

17. The method of claim 16, wherein generating the variant-indicative signal based on the subread analysis of the sequencing reads comprises:
partitioning the sequencing reads into subreads;
mapping the subreads to a corresponding portion of the reference sequence;
detecting the novel adjacencies between at least a portion of the subreads based on at least one of:
a change in a relative position of the portion of the subreads compared to a corresponding original position in the sequencing reads;
a change in an order of the portion of the subreads compared to a corresponding original order in the sequencing reads; or
an alignment of the portion of the subreads to different strands of the reference sequence; and
outputting the novel adjacencies as a discordant junction signal that comprises at least part of the variant-indicative signal.

18. The method of claim 16, wherein generating the variant-indicative signal comprises:
generating a latent breakpoint graph based at least in part on the novel adjacencies by:
defining nodes representing genomic segments from the reference sequence;
defining edges between the nodes, the edges representing adjacencies between the genomic segments in the reference sequence; and
defining additional edges between the nodes based on the novel adjacencies; and
generating a graph alignment by aligning the sequencing data to the latent breakpoint graph.

19. The method of claim 18, further comprising:
generating an initial alignment by aligning the sequencing data to the reference sequence;
for each read of the sequencing data, selecting one of the graph alignment or the initial alignment based on respective alignment scores; and
generating an updated alignment having the selected one of the graph alignment or the initial alignment for each read of the sequencing data.

20. The method of claim 16, wherein generating the variant call comprises:
comparing the variant-indicative signal to the reference sequence to identify genomic differences between the sequencing data and the reference sequence;
for each identified genomic difference:
determining a type of variant based on a size and a type of the identified genomic difference, wherein the type of variant comprises at least one of a deletion structural variant, an insertion structural variant, an inversion structural variant, a translocation structural variant, a duplication structural variant, a complex structural variant, a single-nucleotide polymorphism, or a short indel; and
determining a genomic position of the identified genomic difference; and
generating a report that includes, for each identified genomic difference, the type of variant, the genomic position, and the size.
